(19) United States
(12) Patent Application Publication
(10) Pub. No.: US 2024/0026279 A1
(43) Pub. Date: Jan. 25, 2024

(54) METHODS AND COMPOSITIONS FOR PRODUCING RECOMBINANT COMPONENTS FOR USE IN FOOD AND OTHER PRODUCTS

(71) Applicant: Perfect Day, Inc., Berkeley, CA (US)

(72) Inventors: TIMOTHY GEISTLINGER, Oakland, CA (US); HEATHER JENSEN, Oakland, CA (US); RAVIRAJSINH JHALA, Castro Valley, CA (US); HENDRIK MEERMAN, Encinitas, CA (US); BALAKRISHNAN RAMESH, Berkeley, CA (US); TIMOTHY SCOTT JOHNSON, Oakland, CA (US); VINCENT WEI-XIANG WU, Berkeley, CA (US); FRANCESCA MANEA, Oakland, CA (US); TY WAGONER, Oakland, CA (US)

(21) Appl. No.: 18/037,053

(22) PCT Filed: Nov. 15, 2021
(86) PCT No.: PCT/US21/59413

§ 371 Date: May 15, 2023

Related U.S. Application Data
(60) Provisional application No. 63/113,729, filed on Nov. 13, 2020.
(60) Provisional application No. 63/175,278, filed on Apr. 15, 2021.

(51) Int. Cl. C12N 1/14; A23J 1/20

(52) U.S. Cl. CPC C12N 1/145; A23J 1/20; C12N 2501/70; C12R 2001/885

(57) ABSTRACT

The present disclosure relates to methods and compositions for producing a recombinant component for use in a food or other product. The present disclosure further relates to compositions comprising the recombinant component produced by such methods and compositions.

### RELATED APPLICATIONS

This application claims priority to U.S. Provisional Patent Application Ser. No. 63/113,729, filed on Nov. 13, 2020, and to U.S. Provisional Patent Application Ser. No. 63/175,278, filed on Apr. 15, 2021, which are incorporated herein by reference, in their entireties.

### FIELD OF THE INVENTION

The present disclosure relates generally to methods and compositions for producing a recombinant component for use in a food or other product. The present disclosure further relates generally to compositions comprising the recombinant component produced by such methods and compositions.

### BACKGROUND OF THE INVENTION

Animal-derived food products (e.g., meat, milk, egg) are popular sources of nutrition. They comprise high-quality protein, essential minerals (e.g., calcium, phosphorus, zinc, magnesium), and vitamins (e.g., riboflavin, vitamin A, vitamin B12). In addition, many such food products possess advantageous functional characteristics that permit production of a wide variety of derivative food products (e.g., yogurt, cheese, cream, ice cream, butter, mayonnaise).

However, animal-derived food products comprise components (e.g., lactose, allergens, saturated fats, cholesterol) that can cause unhealthy reactions in humans. Moreover, production of these food products involves animal husbandry, which has a significant impact on animal welfare and the environment, and which bears the potential for contamination with pesticide residues, heavy metals, aflatoxin M1, and pathogens.

These concerns have fueled development of alternatives to animal-derived food and other products (e.g., cosmetics, personal care products). Some such alternatives comprise plant-derived components (e.g., proteins, lipids, vitamins). Increasingly, however, alternatives to animal-derived food and other products are produced from components (e.g., proteins, lipids) that are produced recombinantly (e.g., using recombinant host cells).

The use of recombinant components in food and other products poses new problems. One such problem is that food and other products produced from recombinant components frequently contain a significant amount of such recombinant components (more than was typical in previous products in which recombinant components were utilized), and that the use of large amounts of recombinant components may be impacted by other, sometimes undesired, components that are simultaneous produced by the recombinant host cells from which the recombinant components are obtained, and that potentially co-purify with the recombinant component.

One such other component are enzymes with activity that leads to the release of free fatty acids (FFAs; i.e., FFA releasing enzymes). FFA releasing enzymes can hydrolyze bonds in diglycerides, triglycerides, phospholipids, lipoproteins, and other molecules to release FFAs. Substrates for FFA releasing enzymes are comprised in a variety of food and other products in which recombinant components could be used. In some such food and other products, the release of FFAs can have detrimental effects, by, for example, producing rancid aroma and/or taste, interfering with formation of emulsions, having undesirable effects on texture, interacting with essential nutrients (e.g., vitamins) and thereby decreasing nutritional content, and reducing shelf-life. Production of FFAs in a food and other product may also have beneficial effects, by, for example, producing desired flavor and/or aroma profiles (e.g., flavor profiles of aged cheese), or making enzyme-modified cheese for use in processed cheese. Challenges have thus to be overcome, particularly with respect to FFA releasing enzyme activities, in the production, processing, and use of recombinant components for production of alternatives to animal-derived food and other products, such that, for example production of rancid aroma and taste is delayed, emulsion formation is not impacted, nutritional content is maintained, texture is not modulated, and shelf-life is not shortened; and/or desired flavor and/or aroma profiles are produced.

Therefore, there exists a need for methods by which alternatives to animal-derived food and other products can be produced from recombinant components, as well as for compositions used in and obtained from such methods.

### INCORPORATION BY REFERENCE

All publications, patents, patent applications, sequences, database entries, scientific publications, and other references mentioned herein are incorporated by reference in their entireties to the same extent as if each individual publication, patent, patent application, sequence, database entry, scientific publication, or other reference was specifically and individually indicated to be incorporated by reference. To the extent the material incorporated by reference contradicts or is inconsistent with the present disclosure, the present disclosure, including definitions, will supersede any such material.

### SUMMARY OF THE INVENTION

In various aspects, provided herein is a recombinant host cell capable of producing a recombinant component, wherein the recombinant host cell comprises a modulated production and/or activity of a FFA releasing enzyme compared to production and/or activity of the FFA releasing enzyme comprised in a corresponding recombinant host cell.

The recombinant host cell of paragraph [0010], wherein the FFA releasing enzyme comprises or consists of a FFA releasing enzyme selected from FFA releasing enzymes comprising UniProt sequence #G0RH85, G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, G0RVD2, or G0R8A6, or homologs thereof, or combinations thereof.

The recombinant host cell of paragraph [0010], wherein the FFA releasing enzyme comprises or consists of a FFA releasing enzyme selected from FFA releasing enzymes comprising UniProt sequence #G0RGQ0, G0RH85, G0RMI3, G0RLH4, G0RIU1, G0RBM4, G0R9D1, G0RFR3, G0RG60, G0R6T6, G0R8N5, G0RBJ0, G0RRQ4, G0REZ4, G0RIJ9, G0R6X2, G0RJY0, G0RR42, G0RW77, G0RQJ5, G0RFT3, G0R810, G0RI29, G0RL87, G0RLL0, G0RGD5, or G0RKH7, or homologs thereof, or combinations thereof.

The recombinant host cell of paragraph [0010], wherein the FFA releasing enzyme comprises or consists of a FFA releasing enzyme selected from FFA releasing enzymes comprising UniProt sequence #G0RGQ0, G0RLH4, or G0RMI3, or homologs thereof, or combinations thereof.

The recombinant host cell of paragraph [0010], wherein the FFA releasing enzyme comprises or consists of a FFA releasing enzyme selected from FFA releasing enzymes comprising UniProt sequence #G0RH85 or homologs thereof, or combinations thereof.

The recombinant host cell of paragraph [0010], wherein the FFA releasing enzyme comprises or consists of a FFA releasing enzyme selected from FFA releasing enzymes comprising UniProt sequence #G0RGQ0 or homologs thereof, or combinations thereof.

The recombinant host cell of paragraph [0010], wherein the FFA releasing enzyme comprises or consists of a FFA releasing enzyme selected from FFA releasing enzymes comprising UniProt sequence #G0RLH4 or homologs thereof, or combinations thereof.

The recombinant host cell of paragraph [0010], wherein the FFA releasing enzyme comprises or consists of a FFA releasing enzyme selected from FFA releasing enzymes comprising UniProt sequence #G0RMI3 or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; and a second FFA releasing enzyme selected from FFA releasing enzymes comprising UniProt sequence #G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, R0RVD2, or G0R8A6, or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; and a second FFA releasing enzyme selected from FFA releasing enzymes comprising UniProt sequence #G0RGQ0, G0RH85, G0RMI3, G0RLH4, G0RIU1, G0RBM4, G0R9D1, G0RFR3, G0RG60, G0R6T6, G0R8N5, G0RBJ0, G0RRQ4, G0REZ4, G0RIJ9, G0R6X2, G0RJY0, G0RR42, G0RW77, G0RQJ5, G0RFT3, G0R810, G0RI29, G0RL87, G0RLL0, G0RGD5, or G0RKH7, or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; and a second FFA releasing enzyme selected from FFA releasing enzymes comprising UniProt sequence #G0RGQ0, G0RLH4, G0RMI3, G0R707, G0R7K1, G0R810, G0RFT3, G0RG60, G0RGD5, G0RI29, G0RIJ9, G0RKH7, G0RKL4, G0RL87, G0RLL0, G0RLR3, G0RME5, G0RQJ5, G0RRK3, G0RSK7, G0RWT9, or G0RX82, or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; and a second FFA releasing enzyme selected from FFA releasing enzymes comprising UniProt sequence #G0RGQ0, G0RLH4, or G0RMI3, or homologs thereof, or combinations thereof.

The recombinant host cell of paragraph [0010], wherein the FFA releasing enzyme comprises or consists of first FFA releasing enzyme selected from FFA releasing enzymes comprising UniProt sequence #G0RH85 or homologs thereof, or combinations thereof; and a second FFA releasing enzyme selected from FFA releasing enzymes comprising UniProt sequence #G0RMI3 or homologs thereof, or combinations thereof.

The recombinant host cell of paragraph [0010], wherein the FFA releasing enzyme comprises or consists of a first FFA releasing enzyme selected from a first FFA releasing enzyme selected from FFA releasing enzymes comprising UniProt sequence #G0RH85 or homologs thereof, or combinations thereof; and a second FFA releasing enzyme selected from FFA releasing enzymes comprising UniProt sequence #G0RGQ0 or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; and a second FFA releasing enzyme selected from FFA releasing enzymes comprising UniProt sequence #G0RLH4 or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; a second FFA releasing enzyme selected from FFA releasing enzymes comprising UniProt sequence #G0RMI3 or homologs thereof, or combinations thereof; and a third FFA releasing enzyme selected from FFA releasing enzymes comprising UniProt sequence #G0RGQ0 or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; a second FFA releasing enzyme selected from FFA releasing enzymes comprising UniProt sequence #G0RMI3 or homologs thereof, or combinations thereof; and a third FFA releasing enzyme selected from FFA releasing enzymes comprising UniProt sequence #G0RLH4 or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; a second FFA releasing enzyme selected from FFA releasing enzymes comprising UniProt sequence #G0RGQ0 or homologs thereof, or combinations thereof; and a third FFA releasing enzyme selected from FFA releasing enzymes comprising UniProt sequence #G0RLH4 or homologs thereof, or combinations thereof.

The recombinant host cell of paragraph [0010], wherein the FFA releasing enzyme comprises or consists of a first FFA releasing enzyme selected from FFA releasing enzymes comprising UniProt sequence #G0RH85 or homologs thereof, or combinations thereof; a second FFA releasing enzyme selected from FFA releasing enzymes comprising UniProt sequence #G0RMI3 or homologs thereof, or combinations thereof; a third FFA releasing enzyme selected from FFA releasing enzymes comprising UniProt sequence #G0RGQ0 or homologs thereof, or combinations thereof; and a fourth FFA releasing enzyme selected from FFA releasing enzymes comprising UniProt sequence #G0RLH4 or homologs thereof, or combinations thereof.

The recombinant host cell of any of paragraphs [0010-0028], wherein the modulated production and/or activity of the FFA releasing enzyme comprises a decreased production and/or activity of the FFA releasing enzyme.

The recombinant host cell of paragraph [0029], wherein the decreased production and/or activity of the FFA releasing enzyme is a decreased production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell of any of paragraphs [0010-0030], wherein the recombinant host cell is derived from a bacterium, yeast, or filamentous fungus.

The recombinant host cell of paragraph [0031], wherein the filamentous fungus is selected from Aspergillus (e.g., Aspergillus niger), Trichoderma (e.g., Trichoderma reesei, Trichoderma citrinoviride), or Myceliophthora (e.g., Myceliophthora thermophila).

The recombinant host cell of any of paragraphs [0010-0032], wherein the recombinant component is a recombinant protein.

The recombinant host cell of paragraph [0033], wherein the recombinant protein is a recombinant milk protein.

The recombinant host cell of paragraph [0034], wherein the recombinant milk protein is a recombinant casein.

The recombinant host cell of paragraph [0034], wherein the recombinant milk protein is a recombinant whey protein.

The recombinant host cell of paragraph [0034], wherein the recombinant milk protein is derived from cow, human, sheep, goat, or horse.

In various aspects, provided herein is a method for producing a composition that comprises a recombinant component produced by a recombinant host cell capable of producing the recombinant component, wherein the method comprises modulating a production and/or an activity of a FFA releasing enzyme.

The method of paragraph [0038], wherein the recombinant host cell is the recombinant host cell of any of paragraphs [0010-0037].

The method of paragraph [0038], wherein the modulating a production and/or an activity of a FFA releasing enzyme comprises decreasing a production and/or an activity of the FFA releasing enzyme.

The method of paragraph [0040], wherein the decreasing the production and/or activity of the FFA releasing enzyme is a decreasing production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The method of paragraphs [0040] or [0041], wherein the decreasing a production and/or an activity of the FFA releasing enzyme comprises adding to a fermentation broth, preparation, or composition an inhibitor of a FFA releasing enzyme.

The method of paragraph [0042], wherein the inhibitor of the FFA releasing enzyme is an inhibitor of an FFA releasing enzyme that comprises a serine residue in its catalytic domain.

The method of paragraphs [0040] or [0041], wherein the decreasing a production and/or an activity of the FFA releasing enzyme comprises purifying the recombinant component away from the FFA releasing enzyme activity and/or purifying the FFA releasing enzyme away from the recombinant component.

The method of paragraph [0044], wherein the purifying the FFA releasing enzyme away from the recombinant component comprises use of an activity-based protein profiling (ABPP) small-molecule probe.

In various aspects, provided herein is a method for producing a recombinant component, wherein the method comprises fermenting the recombinant host cell of any of paragraphs [0010-0037] in a culture medium under conditions suitable for production of the recombinant component.

The method of paragraph [0046], wherein the method further comprises purifying the recombinant component to obtain a preparation comprising the recombinant component; and/or post-processing the recombinant component.

The method of paragraph [0047], wherein the purifying comprises purifying to obtain a preparation comprising the recombinant component at a purity of greater than 90%.

The method of paragraph [0047], wherein the post-processing comprises spray drying or concentrating the recombinant component to obtain a powder.

In various aspects, provided herein is a composition comprising a recombinant component, wherein the composition is produced by the method of any of paragraphs [0038-0045].

The composition of paragraph [0050], wherein the composition comprises between about 0.1% and about 100% by dry mass of the recombinant component.

The composition of paragraph [0050], wherein the composition is a food product.

The composition of paragraph [0052], wherein the composition is a supplemented food product.

The composition of paragraph [0053], wherein the composition is a supplemented dairy product.

The composition of paragraph [0053], wherein the composition is a supplemented egg product.

The composition of paragraph [0052], wherein the composition is a substitute food product.

The composition of paragraph [0056], wherein the composition is a substitute dairy product.

The composition of paragraph [0057], wherein the composition is a substitute egg product.

The composition of paragraph [0050], wherein the composition is a cosmetic or personal care product.

The composition of any of paragraphs [0050-0059], wherein the recombinant components is a recombinant protein.

The composition of paragraph [0060], wherein the composition is essentially free of any protein other than the recombinant protein.

The composition of paragraph [0060], wherein the composition is essentially free of any recombinant protein other than the recombinant protein.

The composition of paragraph [0060], wherein the recombinant protein is a recombinant milk protein.

The composition of paragraph [0063], wherein the composition is essentially free of any protein other than the recombinant milk protein

The composition of paragraph [0063], wherein the composition is essentially free of any recombinant protein other than the recombinant milk protein.

In various aspects, provided herein is a recombinant host cell that comprises a recombinant expression construct encoding a FFA releasing enzyme, and that comprises an increased production and/or activity of the FFA releasing enzyme compared to a corresponding host cell.

The recombinant host cell of paragraph [0066], wherein the FFA releasing is selected from FFA releasing enzymes comprising UniProt sequence #G0RH85, G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, G0RVD2, or G0R8A6, or homologs thereof.

The recombinant host cell of paragraphs [0066] or [0067], wherein the recombinant host cell is derived from a bacterium, yeast, or filamentous fungus.

The recombinant host cell of paragraph [0068], wherein the filamentous fungus is selected from Aspergillus (e.g., Aspergillus niger), Trichoderma (e.g., Trichoderma reesei, Trichoderma citrinoviride), or Myceliophthora (e.g., Myceliophthora thermophila).

The recombinant host cell of any of paragraphs [0066-0069], wherein the increased production and/or activity of the FFA releasing enzyme is an increase in production and/or activity of at least 50%.

In various aspects, provided herein is a method for producing a FFA releasing enzyme, wherein the method comprises: obtaining the recombinant host cell of any of paragraphs [0066-0070], culturing the recombinant host cell in a culture medium under conditions suitable for production and/or secretion of the FFA releasing enzyme, and optionally purifying the FFA releasing enzyme.

### BRIEF DESCRIPTION OF THE FIGURES

Example embodiments will be described and explained with additional specificity and detail through the use of the accompanying drawings in which:

FIGS. 1A and 1B provide box-and-whisker plots showing results of RNAseq analyses of Trichoderma reesei host cells producing recombinant protein (i.e., recombinant β-lactoglobulin) for presence of G0RMI3, G0RGQ0, and G0RLH4 transcripts, wherein FIG. 1B provides a detailed view of the G0RGQ0 plot shown in FIG. 1A, in accordance with various embodiments of the present invention. FPKMs=Fragments per kilobase of transcript per million mapped reads.

FIG. 2 is a bar plot showing para-phenyl (pNP) acyl ester hydrolyzing activity comprised in recombinant protein (i.e., recombinant β-lactoglobulin) preparations in absence (Sample 1) or presence (Sample 2) of Thermo ActivX TAMRA-FP fluorophosphonmie (inhibitor of hydrolases comprising a serine residue in their catalytic domain), showing removal of FFA releasing activity according to various representative embodiments of the present invention. Activity unit U/g=micromole pNP formation per hour per gram of recombinant protein; Acyl groups: C4=butyrate, C8=octanoate, C12=laurate, and C16=palmitate.

FIG. 3 is a map of a recombinant vector used for production of a recombinant host cell capable of producing a G0RGQ0, G0RLH4, or G0RMI3 protein, in accordance with various representative embodiments of the present invention.

FIG. 4 is a map of a recombinant vector used for production of a recombinant host cell capable of producing a G0RGQ0, G0RLH4, or G0RMI3 protein, in accordance with various representative embodiments of the present invention.

FIG. 5A shows a Western blot of recombinant G0RMI3 protein comprised in fermentation broths of 4 independent recombinant Pichia pastoris transformants, FIG. 5B shows an SDS PAGE gel of recombinant G0RGQ0 produced by 3 independent recombinant Trichoderma reesei transformants, and FIG. 5C shows an SDS PAGE gel of recombinant G0RLH4 produced by 3 independent recombinant Trichoderma reesei transformants, in accordance with various representative embodiments of the present invention.

FIG. 6 are photographs of UV-illuminated wells of 24-well plates comprising rhodamine B in presence (boxed) or absence (not boxed) of G0RMI3, G0RGQ0, and G0RLH4 proteins, in accordance with various representative embodiments of the present invention.

FIG. 7 is a map of a targeting vector used for production of a recombinant host cell comprising an eliminated FFA releasing activity of a G0RH85, G0RMI3, G0RGQ0, and/or G0RLH4 protein, in accordance with various representative embodiments of the present invention.

### DETAILED DESCRIPTION OF THE INVENTION

The subsequent discussion of the invention is presented for purposes of illustration and description, and is not intended to limit the scope of the invention to the embodiments disclosed herein. As such, variations and modifications of the disclosed embodiments are within the scope of the invention, e.g., as may be within the skill and knowledge of those in the art after understanding the present disclosure. It is intended to obtain rights which include alternative embodiments to the extent permitted, including alternate, interchangeable and/or equivalent structures, functions, ranges or steps to those disclosed herein, and without intending to publicly dedicate any patentable subject matter. Unless defined otherwise, all technical and scientific terms used herein have the same meaning as is commonly understood by one of ordinary skill in the art to which this disclosure pertains. Further, unless otherwise required by context, singular terms shall include the plural, and plural terms shall include the singular.

### Definitions

The terms “a” and “an” and “the” and similar references as used herein refer to both the singular and the plural (e.g., meaning “at least one” or “one or more”), unless otherwise indicated herein or clearly contradicted by context. For example, the term “a compound” is synonymous with the terms “at least one compound” and “one or more compounds”, and may refer to a single compound or to a plurality of compounds, including mixtures thereof.

The term “and/or” as used herein refers to multiple components in combination with or exclusive of one another. For example, “x, y, and/or z” may refer to “x” alone, “y” alone, “z” alone, “x, y, and z”, “(x and y) or z”, “(x and z) or y”, “(y and z) or x”, “x and y” alone, “x and z” alone, “y and z” alone, or “x or y or z”.

The term “at least” or “one or more” as used herein refers to one, two, three, four, five, six, seven, eight, nine, ten, at least one, at least two, at least three, at least four, at least five, at least six, at least seven, at least eight, at least nine, at least ten, or more, or all of the elements subsequently listed.

The term “encoding” as used herein in context of a polynucleotide refers to a polynucleotide that comprises a coding sequence that when placed under the control of appropriate regulatory sequences is transcribed into mRNA that may be translated into a polypeptide. A coding sequence generally starts at a start codon (e.g., ATG) and ends at a stop codon (e.g., UAA, UAG and UGA). A coding sequence may contain a single open reading frame, or several open reading frames (e.g., separated by introns).

The term “endogenous” as used herein refers to what is natively present in the context described. When used in reference to a protein that is produced by a cell, the term implies that the protein is natively produced by the cell. When used in reference to a polynucleotide that is comprised in a cell, the term implies that the polynucleotide is natively comprised in the cell (e.g., is present in the native cell; or is situated in the same genomic location in the native cell).

The terms “FFA releasing enzyme activity” or “activity of a FFA releasing enzyme” as used herein refer to the activity of an enzyme that can hydrolyze a bond (e.g., hydrolase an ester bond) that leads to the release of a free fatty acid (FFA). FFA releasing enzymes are designated with enzyme commission number (EC number) 3.1. The terms are used interchangeably herein.

The term “essentially free of” as used herein refers to the indicated component being either not detectable in the indicated composition by common analytical methods, or to the indicated component being present in such trace amount as to not be functional. The term “functional” as used in this context refers to not materially contributing to properties of the composition comprising the trace amount of the indicated component, or to not having material activity (e.g., chemical activity, enzymatic activity) in the indicated composition comprising the trace amount of the indicated component, or to not having health-adverse effects upon use or consumption of the composition comprising the trace amount of the indicated component. The term “materially contributing” as used herein refers to the indicated component contributing to an attribute of a composition to such extent that in the absence of the component (e.g., in a reference composition that is identical to the composition except that it lacks the indicated component) the attribute is at least 10%, at least 20%, at least 30%, at least 40%, or at least 50% less present/active/measurable.

The term “fermentation broth” as used herein refers to a culture comprising a recombinant host cell capable of producing a recombinant component.

The term “filamentous fungus” as used herein refers to an organism from the filamentous form of the subdivision Eumycota and Oomycota (as defined by Hawksworth et al., In, Ainsworth and Bisby's Dictionary of The Fungi, 8th edition, 1995, CAB International, University Press, Cambridge, UK). A filamentous fungus is distinguished from a yeast by its hyphal elongation during vegetative growth.

The term “fungus” as used herein refers to organisms of the phyla Ascomycotas, Basidiomycota, Zygomycota, and Chythridiomycota, Oomycota, and Glomeromycota. It is understood, however, that fungal taxonomy is continually evolving, and therefore this specific definition of the fungal kingdom may be adjusted in the future.

The term “heterologous” as used herein refers to not being natively present in the context described. When used in reference to a protein that is produced by a cell, the term implies that the protein is not natively produced by the cell. When used in reference to a polynucleotide that is comprised in a cell, the term implies that the polynucleotide is not natively comprised in the cell (e.g., is not present in the native cell; or is not situated in the genomic location in the native cell, whether or not the heterologous polynucleotide is itself endogenous (originating from the same cell or progeny thereof) or exogenous (originating from a different cell or progeny thereof)).

The term “homolog” as used herein refers to a protein that comprises an amino acid sequence that is at least 40% (e.g., at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100%) identical to a sequence of amino acids of a similar length (i.e., a length that is within +/−20% of the length of the query amino acid sequence) comprised in a reference protein, and that has a functional property that is similar (e.g., is within 50%, within 40%, within 30%, within 20%, or within 10% of) or identical to that of the reference protein. The term includes polymorphic variants, interspecies homologs (e.g., orthologs), paralogs, and alleles of a protein, as well as variants that are man-made using genetic engineering techniques.

The term “host cell” as used herein refers not only to the particular subject cell but to the progeny of such cell. Because certain modifications may occur in succeeding generations due to either mutation or environmental influences, such progeny may not, in fact, be identical to the subject cell, but are still included within the scope of the term “host cell” as used herein.

The terms “identity” or “identical” in the context of two or more polynucleotide or polypeptide sequences as used herein refer to the nucleotide or amino acid residues that are the same when the two or more polynucleotide or polypeptide sequences, respectively, are aligned for maximum correspondence. Depending on the application, the “identity” can exist over a region of the sequences being compared (e.g., over the length of a functional domain) or over the full length of the sequences. A “region” is considered to be a continuous stretch of at least 6, 9, 14, 19, 24, 29, 34, 39, or more nucleotides, or of at least 2, 6, 10, 14, 18, 22, 26, 30, or more amino acids. For comparison, typically one sequence acts as a reference sequence to which one or more test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Sequence Analysis Software Package of the Genetics Computer Group (GCG), University of Wisconsin Biotechnology Center, which can be used with default parameters), or by visual inspection (see generally Ausubel et al., infra). One example of an algorithm that is suitable for determining percent sequence identity and sequence similarity is the BLAST algorithm (see, for example, Altschul et al. [1990] J. Mol. Biol. 215:403-410; Gish & States. [1993] Nature Genet. 3:266-272; Madden et al. [1996] Meth. Enzymol. 266:131-141; Altschul et al. [1997] Nucleic Acids Res. 25:3389-3402; Zhang 7 Madden. [1997] Genome Res. 7:649-656). Software for performing BLAST analyses is publicly available through the National Center for Biotechnology Information.

The terms “including,” “includes,” “having,” “has,” “with,” or variants thereof as used herein are intended to be inclusive in a manner similar to the term “comprising”.

The term “un-modulated” as used herein in connection with a FFA releasing enzyme activity refers to a lack of alteration of an activity and/or expression of a FFA releasing enzyme (e.g., no change in a concentration of a FFA releasing enzyme or its enzymatic activity towards a substrate).

The term “modulated” as used herein in connection with a FFA releasing enzyme activity refers to any alteration of an activity of a FFA releasing enzyme. Such modulated FFA releasing enzyme activity is typically due either to an increase or decrease in a concentration of a FFA releasing enzyme, or an increase or decrease in an enzymatic activity of a FFA releasing enzyme towards a substrate. For example, the term can refer to a decrease in activity by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%. Alternatively, the term can refer to an increase in activity by at least 5%, at least 10%, at least 15%, at least 20%, at least 25%, at least 50%, at least 75%, at least 100%, at least 150%, at least 200%, at least 300%, at least 400%, at least 500%, at least 600%, at least 700%, at least 800%, at least 900%, or at least 1,000%.

The term “native” as used herein refers to what is found in nature in its unmodified state (e.g., a cell that is not genetically modified by a human, and that is maintained under conditions [e.g., level of oxygenation, pH, salt concentration, temperature, and nutrient (e.g., carbon, nitrogen, sulfur) availability] that are not defined by a human).

The term “operably linked” as used herein refers to an arrangement of elements that allows them to be functionally related. For example, a promoter sequence is operably linked to a protein coding sequence if it controls the transcription of the protein coding sequence, and a secretion signal sequence is operably linked to a protein if the secretion signal sequence directs the protein through the secretion system of a cell. An “operably linked” element may be in contiguous linkage with another element, or act in trans or at a distance to another element. Non-limiting examples of functions that may be operably linked include control of transcription, control of translation, protein folding, and protein secretion.

The term “one or more” as used herein refers to one, at least one, two, three, four, five, six, seven, eight, nine, ten, or more, or all of the elements subsequently listed.

The terms “optional” or “optionally” as used herein refer to a feature or structure being present or not, or an event or circumstance occurring or not. The description includes instances in which a feature or structure is present, instances in which a feature or structure is absent, instances in which an event or circumstance occurs, and instances in which an event or circumstance does not occur.

The terms “plant protein”, “animal protein”, and “microbial protein” as used herein refer to polypeptides that comprise a sequence of at least 20 (e.g., at least 20, at least 30, at least 40, at least 50, at least 60, at least 70, at least 80, at least 90, at least 100, or at least 150, and usually not more than 250) amino acids that is at least 80% (e.g., at least 80%, at least 85%, at least 90%, at least 95%, at least 99%, 100%) identical to a sequence of amino acids in a protein natively found in a plant, animal, or microbe (i.e., unicellular organism, including all bacteria, archaea, unicellular protista, unicellular animals, unicellular plants, unicellular fungi, unicellular algae, protozoa, and chromista), respectively (i.e., a protein that is native to a plant cell, animal cell, or microbial cell, respectively).

The term “polynucleotide” as used herein refers to a polymeric form of at least 2 (e.g., at least 5, at least 10, at least 20, at least 30, at least 40, at least 50, at least 100, at least 500, at least 1,000) nucleotides. The term includes both sense and antisense strands of DNA molecules (e.g., cDNA, genomic DNA, synthetic DNA) and RNA molecules (e.g., mRNA, synthetic RNA), as well as analogs of DNA or RNA containing non-natural nucleotide analogs, non-native internucleoside bonds, and/or chemical modifications. A polynucleotide may be modified chemically or biochemically or may contain non-natural or derivatized nucleotide bases. Such modifications include, for example, labels; methylation; substitution of one or more of the naturally occurring nucleotides with an analog; internucleotide modifications such as uncharged linkages (e.g., methyl phosphonates, phosphotriesters, phosphoramidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Examples of modified nucleotides are described in the art (see, for example, Malyshev et al. 2014. Nature 509:385; Li et al. 2014. J. Am. Chem. Soc. 136:826). Also included are synthetic molecules that mimic polynucleotides in their ability to bind to a designated sequence via hydrogen bonding or other chemical interaction. Such molecules are known in the art and include, for example, molecules in which peptide linkages substitute for phosphate linkages in the backbone of the molecule. Other modifications may include, for example, analogs in which the ribose ring contains a bridging moiety or other structure such as the modifications found in “locked” polynucleotides. A polynucleotide may be in any topological conformation. For instance, a polynucleotide may be single-stranded, double-stranded, triple-stranded, quadruplexed, partially double-stranded, branched, hairpinned, circular, or in a padlocked conformation. The term “polynucleotide sequence” as used herein refers to a sequence of nucleotides that are comprised in a polynucleotide or of which a polynucleotide consists.

The terms “polypeptide” and “protein” as used herein can be interchanged, and refer to a naturally-occurring or a naturally not occurring polymeric form of at least 2 (e.g., at least 5, at least 10, at least 20, at least 30, at least 40, at least 50, at least 100) amino acids. A “polypeptide” or “protein” may have an active structure or lack a functional structure, comprise coded and/or non-coded amino acids, comprise amino acids that occur in nature and/or amino acids that do not occur in nature, comprise chemically modified and/or biochemically modified and/or derivatized amino acids, comprise unmodified and/or modified peptide backbones, and/or be monomeric (i.e., having a single chain) or polymeric (i.e., having of two or more chains, which may be covalently or non-covalently associated). The term “amino acid sequence” as used herein refers to a sequence of amino acids that is comprised in a “polypeptide” or “protein”, or of which a “polypeptide” or “protein” consists.

The term “preparation” as used herein refers to a preparation obtained upon separation of a recombinant component from one or more other components of a fermentation broth. For example, a preparation may be a clarified fermentation broth (i.e., a fermentation broth from which cells and cell debris were removed).

The term “promoter sequence” as used herein refers to a polynucleotide that directs transcription of a downstream polynucleotide in a cell. A promoter sequence may include necessary nucleotides near the start site of transcription, such as, in the case of a polymerase II type promoter, a TATA element. A promoter sequence may also optionally include distal enhancer or repressor elements, which may be located as much as several thousand base pairs from the start site of transcription.

The term “purifying” or “purified” or “isolating” or “isolated” as used herein refers to a component being substantially separated from chemicals, cellular components, and cells (e.g., cell walls, membrane lipids, chromosomes, other proteins, other cells in an organism) of the source from which the component originated. The component may be at least 60% pure, e.g., greater than 65%, 70%, 75%, 80%, 85%, 90%, 95%, or 99% pure. The term does not require (albeit allows) that the component be separated from all chemicals, cellular components, and cells.

The term “recombinant component” as used herein refers to a component that is produced recombinantly (i.e., is produced in a recombinant host cell, or is synthesized from a recombinant polynucleotide). Non-limiting examples of recombinant components include recombinant proteins (e.g., microbial proteins, plant proteins (e.g., pea protein (e.g., legumin, vicillin, covicillin), potato protein (e.g., tuberin, protease inhibitor notate II)), animal proteins (e.g., structural proteins (e.g., collagen, tropoelastin, elastin), milk proteins, egg proteins (e.g., ovalbumin, herein ovomucoid, ovalbumin, ovotransferrin, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y)), lipids, carbohydrates, small molecules, food additives (e.g., sweetening agents), food supplements (e.g., vitamins), neutraceuticals, and probiotics.

The term “recombinant protein” as used herein refers to a protein that is produced in a recombinant host cell, or to a protein that is synthesized from a recombinant polynucleotide.

The term “recombinant host cell” as used herein refers to a host cell that comprises a recombinant polynucleotide. Thus, for example, a recombinant host cell may produce a polynucleotide or polypeptide not found in the native (non-recombinant) form of the host cell, or a recombinant host cell may produce a polynucleotide or polypeptide at a level that is different from that in the native (non-recombinant) form of the host cell. It should be understood that such term is intended to refer not only to the particular subject cell but also to the progeny of such a cell. Because certain modifications may occur in succeeding generations due to either mutation or environmental influences, such progeny may not be identical to the subject cell, but are still included within the scope of the term “recombinant host cell” as used herein. A recombinant host cell may be an isolated cell or cell line grown in culture or may be a cell which resides in a living tissue or organism.

The term “recombinant polynucleotide” as used herein refers to a polynucleotide that is removed from its naturally occurring environment, or a polynucleotide that is not associated with all or a portion of a polynucleotide abutting or proximal to the polynucleotide when it is found in nature, or a polynucleotide that is operatively linked to a polynucleotide that it is not linked to in nature, or a polynucleotide that does not occur in nature, or a polynucleotide that contains a modification that is not found in that polynucleotide in nature (e.g., insertion, deletion, or point mutation introduced artificially, e.g., by human intervention), or a polynucleotide that is integrated into a chromosome at a heterologous site. The term may be used, e.g., to describe cloned DNA isolates, or a polynucleotide comprising a chemically synthesized nucleotide analog. A polynucleotide is also considered “recombinant” if it contains a genetic modification that does not naturally occur. For instance, an endogenous polynucleotide is considered a “recombinant polynucleotide” if it contains an insertion, deletion, or substitution of one or more nucleotides that is introduced artificially (e.g., by human intervention). Such modification can introduce into the polynucleotide a point mutation, substitution mutation, deletion mutation, insertion mutation, missense mutation, frameshift mutation, duplication mutation, amplification mutation, translocation mutation, or inversion mutation. The term includes a polynucleotide in a host cell's chromosome, as well as a polynucleotide that is not in a host cell's chromosome (e.g., a polynucleotide that is comprised in an episome). A recombinant polynucleotide in a host cell or organism may replicate using the in vivo cellular machinery of the host cell; however, such recombinant polynucleotide, although subsequently replicated intracellularly, is still considered recombinant for purposes of this invention.

The term “regulatory element” as used herein refers a polynucleotide sequence that mediates, modulates, or regulates expression (e.g., transcription, post-transcriptional events, translation) of a polynucleotide to which the regulatory element is operably linked. Non-limiting examples of regulatory elements include promoter sequences, termination sequences, transcriptional start sequences, translational start sequences, translation stop sequences, enhancer sequences, activator sequences, response elements, protein recognition sites, inducible elements, protein binding sequences, 5′ and 3′ untranslated regions, upstream activation sequences (UAS), introns, operators (i.e., sequences of nucleic acids adjacent to a promoter that comprise a protein-binding domain where a repressor protein can bind and reduce or eliminate activity of the promoter), efficient RNA processing signals (e.g., splicing signals, polyadenylation signals), sequences that stabilize cytoplasmic mRNA, sequences that enhance translation efficiency (e.g., ribosome binding sites [e.g., Shine-Dalgarno sequences]), sequences that enhance protein stability, sequences that enhance protein secretion, and combinations thereof.

The terms “secrete,” “secretion”, and “secreted” as used herein refer to the process of export of a protein across a cellular membrane and/or cell wall of a cell that produces the protein to an extracellular milieu. As provided herein, such secretion may occur actively or passively.

The term “secretion signal” as used herein refers to a peptide that is operably linked to the N-terminus of a protein, and that mediates the delivery of the protein via the intracellular secretory pathway of a host cell in which the protein is produced (i.e., synthesized) to the exterior of the host cell. Typically, operable linkage of a recombinant protein with a secretion signal requires removal of a start codon of the polynucleotide sequence encoding the recombinant protein.

The term “two or more” as used herein refers to two, three, four, five, six, seven, eight, nine, ten, or more, or all of the elements subsequently listed.

The term “vector” as used herein refers to a nuclei acid that can carry a polynucleotide sequence to be introduced into a host cell. Non-limiting examples of vectors include cloning vectors, expression vectors, shuttle vectors, plasmids, phage particles, viral vectors, cosmids, bacterial artificial chromosomes (BACs), yeast artificial chromosomes (YACs), virus particles (e.g., comprising heterologous polynucleotides), DNA constructs (e.g., produced by cloning or PCR amplification), and linear double-stranded molecules (e.g., PCR fragments). Certain vectors are capable of autonomous replication in a host cell into which they are introduced (e.g., vectors having an origin of replication which functions in the host cell). Other vectors may be integrated into the genome of a host cell upon introduction into the host cell, and are thereby replicated along with the host genome.

The term “yeast” as used herein refers to any organism of the order Saccharomycetales. Vegetative growth of yeast is by budding/blebbing of a unicellular thallus, and carbon catabolism may be fermentative.

Recitation of ranges of values herein are merely intended to serve as a shorthand method of referring individually to each separate value (fractional or integral) falling within the range inclusive of the recited minimum and maximum value, unless otherwise indicated herein, and each separate value is incorporated into the specification as if it were individually recited herein. Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of less than or equal to 10. It should further be understood that all ranges and quantities described below are approximations and are not intended to limit the invention.

It should be understood that in any method disclosed herein the order of steps or order for performing certain actions is immaterial so long as the invention remains operable. Moreover, two or more steps or actions may be conducted simultaneously.

### Method for Producing Composition Comprising Recombinant Component

In various aspects, provided herein is a method for producing a composition that comprises a recombinant component (e.g., any of the recombinant components disclosed herein) produced by a recombinant host cell capable of producing the recombinant component, wherein the method comprises modulating an activity of a FFA releasing enzyme (e.g., activity of any one FFA releasing enzyme disclosed herein or activities of any combination of two or more FFA releasing enzymes disclosed herein).

Modulating an activity of a FFA releasing enzyme in a method provided herein may occur in any single step or in any combination of two or more steps that provide for a modulated FFA releasing enzyme activity. Non-limiting examples of suitable steps include: i) culturing the recombinant host cell under fermentation conditions suitable for modulating a FFA releasing enzyme activity; ii) modulating a FFA releasing enzyme activity in a fermentation broth, a preparation, or a composition; iii) purifying the recombinant component away from a FFA releasing enzyme activity produced by the recombinant host cell and/or purifying a FFA releasing enzyme activity away from the recombinant component; and/or iv) obtaining a recombinant component produced by a recombinant host cell that comprises a modulated FFA releasing enzyme activity.

### Fermentation Conditions Suitable for Modulating FFA Releasing Enzyme Activity

The method according to any of the above may comprise: a) obtaining a recombinant host cell that is capable of producing a recombinant component; and b) culturing the recombinant host cell in a culture medium under fermentation conditions suitable for production and/or secretion of the recombinant component and for modulating a FFA releasing enzyme activity.

Conditions suitable for modulating a FFA releasing enzyme activity may be, for example, conditions under which the recombinant host cell modulates its production of the FFA releasing enzyme activity. Non-limiting examples of such conditions include a suitable pH, a suitable temperature, a suitable feed rate, a suitable pressure, a suitable fluid shear force, a suitable type and/or amount of a nutrient (e.g., a suitable carbon content, a suitable nitrogen content, a suitable phosphorus content), a suitable type and/or amount of a culture supplement, a suitable type and/or amount of a trace metal, and/or a suitable level of oxygenation.

A suitable pH may be a pH of between 2 and 7.5, 6.5, 6, 5.5, 5, 4.5, 4, 3.5, 3, or 2.5; between 2.5 and 7.5, 6.5, 6, 5.5, 5, 4.5, 4, 3.5, or 3; between 3 and 7.5, 6.5, 6, 5.5, 5, 4.5, 4, or 3.5; between 3.5 and 7.5, 6.5, 6, 5.5, 5, 4.5, 4; between 4 and 7.5, 6.5, 6, 5.5, 5, 4.5; between 4.5 and 7.5, 6.5, 6, 5.5, 5; between 5 and 7.5, 6.5, 6, 5.5; between 5.5 and 7.5, 6.5, 6; between 6 and 7.5, 6.5; between 6.5 and 7.5, or 7; or between 7 and 7.5.

A suitable type of culture supplement may be an anti-foam agent. Non-limiting examples of suitable anti-foam agents include Struktol J 673 A (Schill & Seilacher GmbH, Hamburg, Germany), Industrol DF204 (BASF Canada, Inc., Mississauga, Canada), Polyglycol P-2000 (Dow, Midland, MI), Hodag K-60K (Hodag Chemical Corp., Chicago, IL), and Erol DF6000K (PMC Ouvrie, Carvin, France), ACP 1500 (Dow Chemical Company, Midland, MI), Antifoam 204 (Sigma-Aldrich, St Louis, MO), SAG 471 (Momentive Performance Materials Inc., Waterford, NY), SAG 5693 (Momentive Performance Materials Inc., Waterford, NY), SAG 710 (Momentive Performance Materials Inc., Waterford, NY), SAG 730 (Momentive Performance Materials Inc., Waterford, NY), silicone antifoams, Struktol J647 (Schill & Seilacher, Hamburg, Germany), and sunflower oil.

### Modulating FFA Releasing Enzyme Activity in Fermentation Broth, Preparation, or Composition

The method according to any of the above may comprise: i) obtaining a recombinant host cell that is capable of producing a recombinant component; ii) culturing the recombinant host cell in a culture medium under conditions suitable for production and/or secretion of the recombinant component to obtain a fermentation broth comprising the recombinant component; iii) optionally purifying the recombinant component from the fermentation broth to obtain a preparation comprising the recombinant component; and iv) modulating a FFA releasing enzyme activity in the fermentation broth, preparation, or composition comprising the recombinant component.

A FFA releasing enzyme activity in a fermentation broth, preparation, or composition may be modulated by, for example, adding to the fermentation broth, preparation, or composition a FFA releasing enzyme inhibitor. Non-limiting examples of suitable FFA releasing enzyme inhibitors include synthetic inhibitors (e.g., phosphonates, boronic acid, lipid analogues) and natural inhibitors (e.g., β-lactones (such as valilactone, ebelactone A & B, and vibralactone), manno-oligosaccharides, acetylcholine esterase inhibitors, cholinesterase inhibitors, polyphenols, saponins, panclicins, hesperidin, caulerpenyne, proanthocyanidin, Orlistat (Xenical), carnosic acid, escin, crocin, crocetin, chlorogenic acid, neochlorogenic acid, feruloyquinic acid, e-polylysine, chitosan, chitin), isolated, for example, from sources such as Juniperus communis, Illicium religiosum, Panax japonicus rhizomes, Panax ginseng, Panax quinquefolius, Acanthopanax senticosus, Camellia sinensis var. sinensis, Camellia sinensis var. assamica, Kochia scoparia, Platycodi radix, Salacia reticulate, Nelumbo nucifera, Salix matsudana, Eriochloa villosa, Salacia reticulate, Scabiosa tschiliensis Grun, and Acanthopanax sessiliflorous. In some embodiments, FFA releasing enzyme activity is modulated by addition to a fermentation broth, preparation, or composition of an inhibitor of FFA releasing enzymes that comprise a serine residue in their catalytic domain (e.g., inhibitor Thermo ActivX TAMRA-FP fluorophosphonate). Non-limiting examples of FFA releasing enzymes that comprise a serine residue in their catalytic domain include G0RGQ0, G0RH85, G0RMI3, G0RLH4, G0RIU1, G0RBM4, G0R9D1, G0RFR3, G0RG60, G0R6T6, G0R8N5, G0RBJ0, G0RRQ4, G0REZ4, G0RIJ9, G0R6X2, G0RJY0, G0RR42, G0RW77, G0RQJ5, G0RFT3, G0R810, G0RI29, G0RL87, G0RLL0, G0RGD5, and G0RKH7.

A FFA releasing enzyme activity in a fermentation broth, preparation, or composition also may be modulated by, for example, removing from and/or adding to the fermentation broth, preparation, or composition a cofactor required for activity of a FFA releasing enzyme and/or a cofactor required for activity of a FFA releasing enzyme inhibitor, respectively. Non-limiting examples of such cofactors include metals (e.g., divalent cations, such as calcium), which may be removed, for example, with a chelating agent such as ethylenediaminetetraacetic acid (EDTA).

A FFA releasing enzyme activity in a fermentation broth, preparation, or composition also may be modulated by, for example, thermal or non-thermal processing. Non-limiting examples of thermal processing include pasteurizing and sterilizing. Non-limiting examples of non-thermal processing include high pressure pasteurizing (i.e., high-pressure processing, HPP), ultrasonicating, pulse electric field processing, and irradiating. In some embodiments, a FFA releasing enzyme activity in a fermentation broth, preparation, or composition is essentially eliminated or reduced by incubating the fermentation broth, preparation, or composition at high temperature for a relatively short period of time (e.g., at a temperature of between 85° C. and 90° C. for between 5 and 10 min).

Purifying Recombinant Component Away from FFA Releasing Enzyme Activity

The method according to any of the above may comprise: i) obtaining a recombinant host cell capable of producing a recombinant component; ii) culturing the recombinant host cell in a culture medium under conditions suitable for production and/or secretion of the recombinant component to obtain a fermentation broth comprising the recombinant component; and iii) purifying the recombinant component away from a FFA releasing enzyme activity and/or purifying the FFA releasing enzyme away from the recombinant component to obtain a preparation comprising the recombinant component.

Purifying a recombinant component away from a FFA releasing enzyme activity may be accomplished on the basis of any one property that differentiates the recombinant component from a FFA releasing enzyme, or on the basis of a combination of two or more such properties used in succession (e.g., separation based on charge followed by separation based on hydrophobicity, separation based on hydrophobicity followed by separation based on pH stability) or in parallel (e.g., separation based on pH stability and thermolability/thermostability, separation based on pH stability and affinity to a specific molecule, separation based on solubility and pH stability and/or thermolability/thermostability and/or pI).

For example, a fermentation broth or preparation comprising the recombinant component may be heated to a temperature at which a FFA releasing enzyme is denatured and precipitates out of solution but at which the recombinant component remains structurally intact and soluble. The precipitated FFA releasing enzyme may subsequently be separated from the soluble recombinant component by any suitable method, including 1-g sedimentation, accelerated sedimentation via centrifugation, and/or a variety of filtration techniques, including but not limited to depth filtration or tangential flow filtration, using filter pads, sheets, or membranes.

As a further example, a suitable chromatographic support may be added to a fermentation broth or preparation comprising the recombinant component, and conditions (e.g., pH and/or ionic strength) may be adjusted such that the FFA releasing enzyme or the recombinant component (but not both) binds to the chromatographic support (e.g., based on charge or hydrophobicity), leaving behind the soluble recombinant component or FFA releasing enzyme in an unbound portion, respectively. Non-limiting examples of suitable chromatographic supports include phenyl sepharose, butyl sepharose, and octyl sepharose. The chromatographic support with bound FFA releasing enzyme or recombinant component may subsequently be separated from the soluble recombinant component or FFA releasing enzyme, respectively, by any suitable method known in the art, including but not limited to 1-g sedimentation, centrifugation, or filtration. Alternatively, the chromatographic support may be a stationary support (e.g., adsorbent in a column) through which the fermentation broth or preparation comprising the recombinant component is made to travel, and the recombinant component is obtained in the unbound portion whereas the FFA releasing enzyme binds to the chromatographic support, or the recombinant component is bound to the chromatographic support whereas and the FFA releasing enzyme remains in the unbound portion, and the bound recombinant component is subsequently released from the chromatograph support by adjusting conditions.

As a further example, a counterion or ion-exchange resin or sodium acid salt may be added to a fermentation broth or preparation comprising the recombinant component, and the pH and/or ionic strength of the fermentation broth or preparation may be adjusted such that the counterion or ion-exchange resin or sodium acid salt forms a complex with the recombinant component or the FFA releasing enzyme, leaving behind a soluble FFA releasing enzyme or recombinant component, respectively. The complex may subsequently be isolated by any suitable method known in the art, including 1-g sedimentation, accelerated sedimentation via centrifugation, and/or a variety of filtration techniques, including but not limited to depth filtration or tangential flow filtration, using filter pads, sheets, or membranes. In embodiments in which the recombinant component is complexed to the counterion or ion-exchange resin or sodium acid salt, the recombinant component may be extracted from the complex by adjusting conditions (e.g., adjusting pH and/or ionic strength).

As a further example, the pH of a fermentation broth or preparation comprising the recombinant component may be adjusted such that a FFA releasing enzyme is denatured and precipitates out of solution, leaving behind soluble recombinant component. The precipitated FFA releasing enzyme may subsequently be separated from the soluble recombinant component by any suitable method, including but not limited to 1-g sedimentation, accelerated sedimentation via centrifugation, and/or a variety of filtration techniques, including but not limited to depth filtration or tangential flow filtration, using filter pads, sheets, or membranes.

As a further example, an activity-based protein profiling (ABPP) small-molecule probe may be added to a fermentation broth or preparation comprising the recombinant component. The probe may bind to an FFA releasing enzyme that comprises a serine residue in its catalytic domain. Upon binding of the ABPP to an FFA releasing enzyme, the bound FFA releasing enzyme may be immobilized via a second specific affinity binding, which may result in removal of the FFA releasing enzyme from the recombinant component. Non-limiting examples of ABPPs include biotinylated fluorophosphonates (e.g., ActivX™ desthiobiotin-fluorophosphonate serine hydrolase probe (DTB-FP)), wherein the phosphonate moiety covalently may bind to a nucleophilic serine in the catalytic domain of a FFA releasing enzyme, and wherein the biotin moiety may interact with avidinylated agarose and thereby allow for immobilization of the reacted FFA releasing enzyme (e.g., to agarose beads) and physical separation via centrifugation, filtration, and/or other soluble-insoluble separation method.

### FFA Releasing Enzyme Activity

A modulated FFA releasing enzyme activity as provided herein (e.g., a modulated FFA releasing enzyme activity comprised in a fermentation broth or preparation comprising a recombinant component, or comprised in a recombinant host cell provided herein capable of producing a recombinant component) may be a modulated activity of any one FFA releasing enzyme or any combination of two or more FFA releasing enzymes.

Non-limiting examples of suitable FFA releasing enzymes include carboxylic ester hydrolases (EC number 3.1.1), phosphoric diester hydrolases (EC number 3.1.4), exoribonucleases (EC number 3.1.13), carboxylesterases (EC number 3.1.1.1), arylesterases (EC number 3.1.1.2), triacylglycerol lipases (EC number 3.1.1.3), phospholipases A2 (EC number 3.1.1.4), lysophospholipases (EC number 3.1.1.5), acetylesterases (EC number 3.1.1.6), acetylcholinesterases (EC number 3.1.1.7), glycerophosphocholine phosphodiesterases (EC number 3.1.4.2), phospholipases C (EC number 3.1.4.3), phospholipases D (EC number 3.1.4.4), phosphoinositide phospholipases C (EC number 3.1.4.11), glycosylphosphatidylinositol phospholipases D (EC number 3.1.4.50), N-acetylphosphatidylethanolamine-hydrolyzing phospholipases D (EC number 3.1.4.54), pectinesterases (EC number 3.1.1.11), gluconolactonases (EC number 3.1.1.17), acylglycerol lipases (EC number 3.1.1.23), 3-oxoadipate enol-lactonases (EC number 3.1.1.24), 1,4-lactonases (EC number 3.1.1.25), galactolipases (EC number 3.1.1.26), phospholipases A1 (EC number 3.1.1.32), lipoprotein lipases (EC number 3.1.1.34), cephalosporin-C deacetylases (EC number 3.1.1.41), carboxymethylenebutenolidases (EC number 3.1.1.45), 2-pyrone-4,6-dicarboxylate lactonases (EC number 3.1.1.57), acetylxylan esterases (EC number 3.1.1.72), feruloyl esterases (EC number 3.1.1.73), cutinases (EC number 3.1.1.74), hormone-sensitive lipases (EC number 3.1.1.79), palmitoyl-protein hydrolases (EC number 3.1.2.22), epoxide hydrolases (EC number 3.3.2.3), ceramidases (EC number 3.5.1.23), leukotriene-A4 hydrolases (EC number 3.3.2.6), hepoxilin-epoxide hydrolase (EC number 3.3.2.7), limonene-1,2-epoxide hydrolases (EC number 3.3.2.8), microsomal epoxide hydrolase (EC number 3.3.2.9), soluble epoxide hydrolase (EC number 3.3.2.10), cholesterol-5,6-oxide hydrolase (EC number 3.3.2.11), fatty acid amide hydrolase (EC number 3.5.1.99), and lipoxygenases (E.C. 1.13.11.).

Non-limiting examples of suitable FFA releasing enzymes include any of the FFA releasing enzymes listed in Table 1, and homologs thereof, as well as FFA releasing enzymes that comprise any one of the InterPro domains listed in Table 2.

TABLE 1
Exemplary FFA Releasing Enzymes (UniProt Sequence IDs)

| | | | | | |
|---|---|---|---|---|---|
| A0A2T4AZ21 | A2QUQ1 | G0R733 | G0RES5 | G0RL78 | G0RSX2 |
| A0A2T4B235 | A2QV27 | G0R765 | G0REU9 | G0RL87 | G0RT28 |
| A0A2T4BBP9 | A2QV39 | G0R7A1 | G0REV0 | G0RLA5 | G0RT33 |
| A0A2T4BCL7 | A2QV40 | G0R7C6 | G0REW1 | G0RLB0 | G0RT48 |
| A0A2T4BFY3 | A2QV44 | G0R7E2 | G0REZ4 | G0RLB7 | G0RT83 |
| A0A2T4BJB5 | A2QVF5 | G0R7I3 | G0RF39 | G0RLF0 | G0RTA0 |
| A0A2T4BJD9 | A2QVJ4 | G0R7I6 | G0RF50 | G0RLF5 | G0RTF8 |
| A0A2T4BNI9 | A2QW25 | G0R7K1 | G0RF55 | G0RLH4 | G0RTH0 |
| A2Q818 | A2QW83 | G0R7K4 | G0RF60 | G0RLJ6 | G0RTI0 |
| A2Q8F7 | A2QX56 | G0R7P7 | G0RFF4 | G0RLL0 | G0RTJ0 |
| A2Q8R7 | A2QX92 | G0R7T1 | G0RFG1 | G0RLR3 | G0RTK4 |
| A2Q8U6 | A2QXD2 | G0R7V3 | G0RFK7 | G0RLW2 | G0RTR6 |
| A2Q9L0 | A2QY19 | G0R810 | G0RFN9 | G0RM14 | G0RTT4 |
| A2QAC4 | A2QYC0 | G0R858 | G0RFR3 | G0RM75 | G0RTU6 |
| A2QAD7 | A2QYF0 | G0R862 | G0RFT3 | G0RME1 | G0RTX4 |
| A2QAH7 | A2QYK5 | G0R882 | G0RFV5 | G0RME5 | G0RUB4 |
| A2QBC9 | A2QYS7 | G0R8A6 | G0RFW1 | G0RMI3 | G0RUF0 |
| A2QBG8 | A2QYU7 | G0R8F3 | G0RFX4 | G0RMI7 | G0RUF6 |
| A2QBH3 | A2QZ17 | G0R8H0 | G0RFY5 | G0RMJ0 | G0RUH2 |
| A2QBH5 | A2QZ72 | G0R8H3 | G0RFZ5 | G0RMK6 | G0RUI0 |
| A2QBH8 | A2QZB2 | G0R8N5 | G0RG04 | G0RMN0 | G0RUL0 |
| A2QBK3 | A2QZB7 | G0R8Z3 | G0RG60 | G0RMS9 | G0RUQ7 |
| A2QBP1 | A2QZE3 | G0R908 | G0RG87 | G0RMU2 | G0RUS8 |
| A2QC75 | A2QZI3 | G0R932 | G0RGA2 | G0RMV2 | G0RUU0 |
| A2QCM0 | A2QZK9 | G0R976 | G0RGD5 | G0RMW2 | G0RUX0 |
| A2QCT1 | A2QZN6 | G0R9B7 | G0RGG2 | G0RMX3 | G0RUY2 |
| A2QE05 | A2QZR0 | G0R9D1 | G0RGK1 | G0RMZ1 | G0RUZ9 |
| A2QE77 | A2QZW3 | G0R9F9 | G0RGL4 | G0RN71 | G0RV27 |
| A2QEH4 | A2QZX0 | G0R9J9 | G0RGN7 | G0RNC1 | G0RV66 |
| A2QEJ2 | A2QZX4 | G0R9K4 | G0RGQ0 | G0RNE8 | G0RV80 |
| A2QEW9 | A2QZY6 | G0R9R2 | G0RGQ7 | G0RNF6 | G0RV93 |
| A2QF42 | A2R032 | G0R9S9 | G0RGR3 | G0RNF8 | G0RV93 |
| A2QF54 | A2R088 | G0R9U0 | G0RGU9 | G0RNI2 | G0RVC2 |
| A2QF64 | A2R098 | G0R9X3 | G0RGV1 | G0RNI4 | G0RVD2 |
| A2QFE9 | A2R0H9 | G0R9X6 | G0RGW7 | G0RNL1 | G0RVJ5 |
| A2QG33 | A2R0P4 | G0RA08 | G0RH18 | G0RNL9 | G0RVK4 |
| A2QG70 | A2R0Z6 | G0RA23 | G0RH28 | G0RNP0 | G0RVK8 |
| A2QGD9 | A2R199 | G0RA68 | G0RH34 | G0RNR1 | G0RVN2 |
| A2QH22 | A2R1N7 | G0RAB1 | G0RH85 | G0RNS6 | G0RVN3 |
| A2QH76 | A2R1P3 | G0RAC5 | G0RHA2 | G0RNS9 | G0RVR8 |
| A2QHB7 | A2R1R5 | G0RAE0 | G0RHA7 | G0RNV2 | G0RVT9 |
| A2QHE2 | A2R1X8 | G0RAQ0 | G0RHB2 | G0RNV2 | G0RVW2 |
| A2QHE9 | A2R234 | G0RAQ2 | G0RHB9 | G0RNX7 | G0RVW9 |
| A2QI32 | A2R256 | G0RAQ9 | G0RHG1 | G0RNX9 | G0RVY3 |
| A2QIA0 | A2R273 | G0RB17 | G0RHG3 | G0RNY4 | G0RVZ5 |
| A2QIE4 | A2R274 | G0RB25 | G0RHH9 | G0RP27 | G0RW00 |
| A2QII0 | A2R215 | G0RB33 | G0RHJ4 | G0RPQ8 | G0RW44 |
| A2QIR3 | A2R2M3 | G0RB46 | G0RHL8 | G0RPU6 | G0RW65 |
| A2QK82 | A2R2W3 | G0RB49 | G0RHQ7 | G0RPX6 | G0RW73 |
| A2QK84 | A2R350 | G0RB72 | G0RHZ4 | G0RQB2 | G0RW77 |
| A2QK90 | A2R496 | G0RBA4 | G0RI29 | G0RQD1 | G0RW78 |
| A2QKQ5 | A2R4Z2 | G0RBA7 | G0RI53 | G0RQE9 | G0RW83 |
| A2QKZ8 | A2R502 | G0RBB4 | G0RIB6 | G0RQF9 | G0RWB8 |
| A2QL89 | A2R5R4 | G0RBC8 | G0RID1 | G0RQG3 | G0RWC3 |
| A2QL90 | A2R5R5 | G0RBG0 | G0RIE0 | G0RQJ5 | G0RWG2 |
| A2QLA0 | A2R5V7 | G0RBH2 | G0RIF0 | G0RQJ7 | G0RWH5 |
| A2QM14 | A2R689 | G0RBJ0 | G0RIF9 | G0RQJ8 | G0RWI8 |
| A2QMI7 | A2R6H5 | G0RBJ9 | G0RIJ9 | G0RQJ9 | G0RWN3 |
| A2QMK5 | A2R6I6 | G0RBK0 | G0RIL6 | G0RQL6 | G0RWP3 |
| A2QN29 | A2R6I8 | G0RBL2 | G0RIU1 | G0RQM0 | G0RWS1 |
| A2QN56 | A2R6L8 | G0RBM4 | G0RIV5 | G0RQN5 | G0RWT9 |
| A2QNF0 | A2R709 | G0RBR4 | G0RIW0 | G0RQW6 | G0RWW6 |
| A2QNW9 | A2R775 | G0RBV8 | G0RIY7 | G0RQY3 | G0RWY5 |
| A2QPC2 | A2R780 | G0RBZ6 | G0RJ62 | G0RR02 | G0RX05 |
| A2QPJ6 | A2R7H4 | G0RC98 | G0RJ65 | G0RR03 | G0RX23 |
| A2QPY4 | A2R7J0 | G0RCA4 | G0RJ76 | G0RR14 | G0RX29 |
| A2QRK1 | A2R835 | G0RCD0 | G0RJ81 | G0RR30 | G0RX49 |
| A2QRK3 | A2R845 | G0RCG3 | G0RJC6 | G0RR42 | G0RX82 |
| A2QRP8 | A2R8C2 | G0RCI8 | G0RJK1 | G0RR69 | G0RX90 |
| A2QS21 | A2R8M8 | G0RCP2 | G0RJP3 | G0RR96 | G0RXG3 |
| A2QS22 | A2R8R3 | G0RCV0 | G0RJQ1 | G0RRB6 | G0RXI5 |
| A2QS33 | A2R8R4 | G0RCV3 | G0RJW8 | G0RRD1 | G0RXL4 |
| A2QS46 | A2R8Z3 | G0RCY9 | G0RJY0 | G0RRG3 | G0RXP9 |
| A2QS56 | A2R9C0 | G0RD04 | G0RK83 | G0RRH7 | G2Q0K1 |
| A2QS66 | A2RAP4 | G0RD16 | G0RK88 | G0RRJ7 | G2Q379 |
| A2QSJ9 | A2RBF9 | G0RD43 | G0RK89 | G0RRK3 | G2QH51 |
| A2QST4 | A5ABC0 | G0RD44 | G0RKE6 | G0RRL3 | G2QL32 |
| A2QSX2 | A5ABC3 | G0RDB2 | G0RKG4 | G0RRQ1 | O32129 |
| A2QSY5 | A5ABE5 | G0RDB5 | G0RKH6 | G0RRQ4 | P37957 |
| A2QT47 | A5ABE6 | G0RDD0 | G0RKH7 | G0RRR1 | P37967 |
| A2QT57 | A5ABE8 | G0RDE1 | G0RKI9 | G0RRS5 | P42969 |
| A2QT66 | A5ABH9 | G0RDJ1 | G0RKJ8 | G0RRS8 | Q79F14 |
| A2QT70 | A5ABK1 | G0RDK5 | G0RKL2 | G0RRV4 | |
| A2QT75 | A5ABZ1 | G0RDN5 | G0RKL4 | G0RRZ7 | |
| A2QT90 | E2PSR0 | G0RDU7 | G0RKL9 | G0RS43 | |
| A2QTI0 | E2PT14 | G0RDV8 | G0RKQ8 | G0RS48 | |
| A2QTI9 | E2PT22 | G0RDW0 | G0RKT8 | G0RSC7 | |
| A2QTZ0 | G0R6T6 | G0RE70 | G0RKT9 | G0RSK7 | |
| A2QUA5 | G0R6X2 | G0REE8 | G0RKW8 | G0RSM4 | |
| A2QUC1 | G0R6X7 | G0REM1 | G0RKX7 | G0RSP4 | |
| A2QUD7 | G0R6Z2 | G0REM4 | G0RL22 | G0RSS3 | |
| A2QUE3 | G0R707 | G0REM9 | G0RL38 | G0RSW4 | |

TABLE 2
InterPro Numbers of Exemplary FFA
Releasing Enzyme Catalytic Domains

| | | | | |
|---|---|---|---|---|
| IPR002168 | IPR017915 | IPR003187 | IPR038885 | IPR008265 |
| IPR033140 | IPR001087 | IPR004961 | IPR039097 | IPR033112 |
| IPR000675 | IPR001531 | IPR007000 | IPR039180 | IPR033113 |
| IPR002641 | IPR003633 | IPR007942 | IPR008947 | IPR001423 |
| IPR002642 | IPR007751 | IPR009613 | IPR036541 | IPR009535 |
| IPR002921 | IPR008475 | IPR010711 | IPR033560 | IPR026605 |
| IPR001711 | IPR010468 | IPR011402 | IPR033562 | IPR028382 |
| IPR001736 | IPR013818 | IPR012354 | IPR033902 | IPR028407 |
| IPR000909 | IPR014815 | IPR016272 | IPR034315 | IPR032075 |
| IPR005592 | IPR015359 | IPR016338 | IPR037737 | IPR032588 |
| IPR006693 | IPR016090 | IPR016445 | IPR038875 | IPR033556 |
| IPR025202 | IPR017913 | IPR016674 | IPR001211 | IPR033903 |
| IPR003140 | IPR017914 | IPR017186 | IPR001981 | IPR033906 |
| IPR011150 | IPR024632 | IPR017766 | IPR002330 | IPR035547 |
| IPR015679 | IPR025920 | IPR017767 | IPR002331 | IPR035669 |
| IPR016555 | IPR029002 | IPR017769 | IPR002333 | IPR000734 |
| IPR021771 | IPR032093 | IPR020009 | IPR002334 | IPR001028 |
| IPR001192 | IPR032341 | IPR025483 | IPR036444 | IPR036691 |
| IPR024884 | IPR016035 | IPR002918 | IPR005152 | IPR015141 |
| IPR001446 | IPR004126 | IPR001885 | IPR000801 | IPR027433 |
| IPR020833 | IPR020834 | IPR013819 | IPR000907 | IPR001246 |

Non-limiting examples of combinations of two or more FFA releasing enzymes include: one or more cutinases and one or more other carboxylic ester hydrolases; one or more lysophospholipases and one or more other carboxylic ester hydrolases; one or more triacylglycerol lipases and one or more other carboxylic ester hydrolases; one or more phospholipases A2 and one or more other carboxylic ester hydrolases; one or more phospholipases C and one or more other carboxylic ester hydrolases; one or more acetylxylanesterase and one or more other carboxylic ester hydrolases; one or more extracellular lipase-like proteins and one or more other carboxylic ester hydrolases; one or more acetylesterases and one or more other carboxylic ester hydrolases; one or more GDSL lipases and one or more other carboxylic ester hydrolases; one or more alpha/beta hydrolases and one or more other carboxylic ester hydrolases; one or more transcription factors regulating expression of a FFA releasing enzyme and one or more other carboxylic ester hydrolases; one or more cutinases and one or more lysophospholipases; one or more cutinases and one or more triacylglycerol lipases; one or more cutinases and one or more phospholipases A2; one or more cutinases and one or more phospholipases C; one or more cutinases and one or more acetylxylanesterases; one or more cutinases and one or more extracellular lipase-like proteins; one or more cutinases and one or more acetylesterases; one or more cutinases and one or more GDSL lipases; one or more cutinases and one or more alpha/beta hydrolases; one or more cutinases and one or more transcription factors regulating expression of a FFA releasing enzyme; one or more lysophospholipases and one or more triacylglycerol lipases; one or more lysophospholipases and one or more phospholipases A2; one or more lysophospholipases and one or more phospholipases C; one or more lysophospholipases and one or more acetylxylanesterases; one or more lysophospholipases and one or more extracellular lipase-like proteins; one or more lysophospholipases and one or more acetylesterases; one or more lysophospholipases and one or more GDSL lipases; one or more lysophospholipases and one or more alpha/beta hydrolases; one or more lysophospholipases and one or more transcription factors regulating expression of a FFA releasing enzyme; one or more triacylglycerol lipases and one or more phospholipases A2; one or more triacylglycerol lipases and one or more phospholipases C; one or more triacylglycerol lipases and one or more acetylxylanesterases; one or more triacylglycerol lipases and one or more extracellular lipase-like proteins; one or more triacylglycerol lipases and one or more acetylesterases; one or more triacylglycerol lipases and one or more GDSL lipases; one or more triacylglycerol lipases and one or more alpha/beta hydrolases; one or more triacylglycerol lipases and one or more transcription factors regulating expression of a FFA releasing enzyme; one or more phospholipases A2 and one or more phospholipases C; one or more phospholipases A2 and one or more acetylxylanesterases; one or more phospholipases A2 and one or more extracellular lipase-like proteins; one or more phospholipases A2 and one or more acetylesterases; one or more phospholipases A2 and one or more GDSL lipases; one or more phospholipases A2 and one or more alpha/beta hydrolases; one or more phospholipases A2 and one or more transcription factors regulating expression of a FFA releasing enzyme; one or more phospholipases C and one or more acetylxylanesterases; one or more phospholipases C and one or more extracellular lipase-like proteins; one or more phospholipases C and one or more acetylesterases; one or more phospholipases C and one or more GDSL lipases; one or more phospholipases C and one or more alpha/beta hydrolases; one or more phospholipases C and one or more transcription factors regulating expression of a FFA releasing enzyme; one or more acetylxylanesterase and one or more extracellular lipase-like proteins; one or more acetylxylanesterase and one or more acetylesterases; one or more acetylxylanesterase and one or more GDSL lipases; one or more acetylxylanesterase and one or more alpha/beta hydrolases; one or more acetylxylanesterase and one or more transcription factors regulating expression of a FFA releasing enzyme; one or more acetylesterases and one or more GDSL lipases; one or more acetylesterases and one or more alpha/beta hydrolases; one or more acetylesterases and one or more transcription factors regulating expression of a FFA releasing enzyme; one or more GDSL lipases and one or more alpha/beta hydrolases; one or more GDSL lipases and one or more transcription factors regulating expression of a FFA releasing enzyme; one or more alpha/beta hydrolases and one or more transcription factors regulating expression of a FFA releasing enzyme; one or more cutinases and one or more lysophospholipases and one or more other carboxylic ester hydrolases; one or more cutinases and one or more triacylglycerol lipases and one or more other carboxylic ester hydrolases; one or more cutinases and one or more phospholipases A2 and one or more other carboxylic ester hydrolases; one or more cutinases and one or more phospholipases C and one or more other carboxylic ester hydrolases; one or more cutinases and one or more acetylxylanesterases and one or more other carboxylic ester hydrolases; one or more cutinases and one or more extracellular lipase-like proteins and one or more other carboxylic ester hydrolases; one or more cutinases and one or more acetylesterases and one or more other carboxylic ester hydrolases; one or more cutinases and one or more GDSL lipases and one or more other carboxylic ester hydrolases; one or more cutinases and one or more alpha/beta hydrolases and one or more other carboxylic ester hydrolases; one or more cutinases and one or more transcription factors regulating expression of a FFA releasing enzyme and one or more other carboxylic ester hydrolases; one or more lysophospholipases and one or more triacylglycerol lipases and one or more other carboxylic ester hydrolases; one or more lysophospholipases and one or more phospholipases A2 and one or more other carboxylic ester hydrolases; one or more lysophospholipases and one or more phospholipases C and one or more other carboxylic ester hydrolases; one or more lysophospholipases and one or more acetylxylanesterases and one or more other carboxylic ester hydrolases; one or more lysophospholipases and one or more extracellular lipase-like proteins; one or more lysophospholipases and one or more acetylesterases and one or more other carboxylic ester hydrolases; one or more lysophospholipases and one or more GDSL lipases and one or more other carboxylic ester hydrolases; one or more lysophospholipases and one or more alpha/beta hydrolases and one or more other carboxylic ester hydrolases; one or more lysophospholipases and one or more transcription factors regulating expression of a FFA releasing enzyme and one or more other carboxylic ester hydrolases; one or more triacylglycerol lipases and one or more phospholipases A2 and one or more other carboxylic ester hydrolases; one or more triacylglycerol lipases and one or more phospholipases C and one or more other carboxylic ester hydrolases; one or more triacylglycerol lipases and one or more acetylxylanesterases and one or more other carboxylic ester hydrolases; one or more triacylglycerol lipases and one or more extracellular lipase-like proteins and one or more other carboxylic ester hydrolases; one or more triacylglycerol lipases and one or more acetylesterases and one or more other carboxylic ester hydrolases; one or more triacylglycerol lipases and one or more GDSL lipases and one or more other carboxylic ester hydrolases; one or more triacylglycerol lipases and one or more alpha/beta hydrolases and one or more other carboxylic ester hydrolases; one or more triacylglycerol lipases and one or more transcription factors regulating expression of a FFA releasing enzyme and one or more other carboxylic ester hydrolases; one or more phospholipases A2 and one or more phospholipases C and one or more other carboxylic ester hydrolases; one or more phospholipases A2 and one or more acetylxylanesterase and one or more other carboxylic ester hydrolases; one or more phospholipases A2 and one or more extracellular lipase-like proteins and one or more other carboxylic ester hydrolases; one or more phospholipases A2 and one or more acetylesterases and one or more other carboxylic ester hydrolases; one or more phospholipases A2 and one or more GDSL lipases, and one or more other carboxylic ester hydrolases; one or more phospholipases A2 and one or more alpha/beta hydrolases and one or more other carboxylic ester hydrolases; one or more phospholipases A2 and one or more transcription factors regulating expression of a FFA releasing enzyme and one or more other carboxylic ester hydrolases; one or more phospholipases C and one or more acetylxylanesterase and one or more other carboxylic ester hydrolases; one or more phospholipases C and one or more extracellular lipase-like proteins; one or more phospholipases C and one or more acetylesterases and one or more other carboxylic ester hydrolases; one or more phospholipases C and one or more GDSL lipases and one or more other carboxylic ester hydrolases; one or more phospholipases C and one or more alpha/beta hydrolases and one or more other carboxylic ester hydrolases; one or more phospholipases C and one or more transcription factors regulating expression of a FFA releasing enzyme and one or more other carboxylic ester hydrolases; one or more acetylxylanesterase and one or more extracellular lipase-like proteins and one or more other carboxylic ester hydrolases; one or more acetylxylanesterase and one or more acetylesterases and one or more other carboxylic ester hydrolases; one or more acetylxylanesterase and one or more GDSL lipases and one or more other carboxylic ester hydrolases; one or more acetylxylanesterase and one or more alpha/beta hydrolases and one or more other carboxylic ester hydrolases; one or more acetylxylanesterase and one or more transcription factors regulating expression of a FFA releasing enzyme and one or more other carboxylic ester hydrolases; one or more acetylesterases and one or more GDSL lipases and one or more other carboxylic ester hydrolases; one or more acetylesterases and one or more alpha/beta hydrolases and one or more other carboxylic ester hydrolases; one or more acetylesterases and one or more transcription factors regulating expression of a FFA releasing enzyme and one or more other carboxylic ester hydrolases; one or more GDSL lipases and one or more alpha/beta hydrolases and one or more other carboxylic ester hydrolases; one or more GDSL lipases and one or more transcription factors regulating expression of a FFA releasing enzyme and one or more other carboxylic ester hydrolases; one or more alpha/beta hydrolases and one or more transcription factors regulating expression of a FFA releasing enzyme and one or more other carboxylic ester hydrolases; one or more cutinases and one or more lysophospholipases and one or more triacylglycerol lipases; one or more cutinases and one or more lysophospholipases and one or more phospholipases A2; one or more cutinases and one or more lysophospholipases and one or more phospholipases C; one or more cutinases and one or more lysophospholipases and one or more acetylxylanesterases; one or more cutinases and one or more lysophospholipases and one or more extracellular lipase-like proteins; one or more cutinases and one or more lysophospholipases and one or more acetylesterases; one or more cutinases and one or more lysophospholipases and one or more GDSL lipases; one or more cutinases and one or more lysophospholipases and one or more alpha/beta hydrolases; one or more cutinases and one or more lysophospholipases and one or more transcription factors regulating expression of a FFA releasing enzyme; one or more cutinases and one or more triacylglycerol lipases and one or more phospholipases A2; one or more cutinases and one or more triacylglycerol lipases and one or more phospholipases C; one or more cutinases and one or more triacylglycerol lipases and one or more acetylxylanesterases; one or more cutinases and one or more triacylglycerol lipases and one or more extracellular lipase-like proteins; one or more cutinases and one or more triacylglycerol lipases and one or more acetylesterases; one or more cutinases and one or more triacylglycerol lipases and one or more GDSL lipases; one or more cutinases and one or more triacylglycerol lipases and one or more alpha/beta hydrolases; one or more cutinases and one or more triacylglycerol lipases and one or more transcription factors regulating expression of a FFA releasing enzyme; one or more cutinases and one or more phospholipases A2 and one or more phospholipases C; one or more cutinases and one or more phospholipases A2 and one or more acetylxylanesterases; one or more cutinases and one or more phospholipases A2 and one or more extracellular lipase-like proteins; one or more cutinases and one or more phospholipases A2 and one or more acetylesterases; one or more cutinases and one or more phospholipases A2 and one or more GDSL lipases; one or more cutinases and one or more phospholipases A2 and one or more alpha/beta hydrolases; one or more cutinases and one or more phospholipases A2 and one or more transcription factors regulating expression of a FFA releasing enzyme; one or more cutinases and one or more phospholipases C and one or more acetylxylanesterases; one or more cutinases and one or more phospholipases C and one or more extracellular lipase-like proteins; one or more cutinases and one or more phospholipases C and one or more acetylesterases; one or more cutinases and one or more phospholipases C and one or more GDSL lipases; one or more cutinases and one or more phospholipases C and one or more alpha/beta hydrolases; one or more cutinases and one or more phospholipases C and one or more transcription factors regulating expression of a FFA releasing enzyme; one or more cutinases and one or more acetylxylanesterases and one or more extracellular lipase-like proteins; one or more cutinases and one or more acetylxylanesterases and one or more acetylesterases; one or more cutinases and one or more acetylxylanesterases and one or more GDSL lipases; one or more cutinases and one or more acetylxylanesterases and one or more alpha/beta hydrolases; one or more cutinases and one or more acetylxylanesterases and one or more transcription factors regulating expression of a FFA releasing enzyme; one or more cutinases and one or more extracellular lipase-like proteins and one or more acetylesterases; one or more cutinases and one or more extracellular lipase-like proteins and one or more GDSL lipases; one or more cutinases and one or more extracellular lipase-like proteins and one or more alpha/beta hydrolases; one or more cutinases and one or more extracellular lipase-like proteins and one or more transcription factors regulating expression of a FFA releasing enzyme; one or more cutinases and one or more acetylesterases and one or more GDSL lipases; one or more cutinases and one or more acetylesterases and one or more alpha/beta hydrolases; one or more cutinases and one or more acetylesterases and one or more transcription factors regulating expression of a FFA releasing enzyme; one or more cutinases and one or more GDSL lipases and one or more alpha/beta hydrolases; one or more cutinases and one or more GDSL lipases and one or more transcription factors regulating expression of a FFA releasing enzyme; and one or more cutinases and one or more alpha/beta hydrolase fold domain-containing proteins and one or more transcription factors regulating expression of a FFA releasing enzyme.

In a method according to any of the above, the fermentation broth or preparation comprising a recombinant component, or the recombinant host cell capable of producing a recombinant component, may be a combination and/or level of FFA releasing enzyme activities that provides a desired profile of FFA releasing enzyme activities (i.e., FFA releasing enzyme activity profile) that is useful for production of a desired composition. In some such embodiments, the FFA releasing enzyme activity profile may be optimized to provide a desired flavor, aroma, texture, emulsification, nutritional content, and/or shelf-life of the composition.

A suitable number and/or combination of FFA releasing enzymes of which activity must be modulated in a method according to any of the above may be identified by methods known in the art. For example, FFA releasing enzymes may be isolated by methods known in the art (e.g., employing affinity chromatography, zymogram assays, gel electrophoresis) and tested in vitro to determine which one or which combination of two or more FFA releasing enzymes provides a substantial amount of degradation of a specific diglycerides, triglyceride, phospholipid, or lipoprotein. Also, recombinant host cells capable of producing a recombinant component (e.g., any recombinant component disclosed herein) may be obtained that comprise a modulated activity in any one or any combination of two or more FFA releasing enzymes, and degree of reduction or elimination of lipid degradation of a composition comprising the recombinant component produced by each such recombinant host cell may be measured by methods known in the art.

FFA releasing enzyme activity may be measured using an enzyme assay. For example, enzyme activity may be determined by production of a colorimetric reaction product or a product that may be detected (e.g., FFAs and/or glycerol produced from an esterase-catalyzed hydrolysis of a triglyceride), using, for example, PAGE gel, spectrophotometer, imaging, UV/Vis, light, and HPLC.

A modulated FFA releasing enzyme activity according to any of the above may comprise or consist of a modulated production and/or activity of a FFA releasing enzyme selected from the group consisting of FFA releasing enzymes comprising UniProt sequence #G0RH85, G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, G0RVD2, and G0R8A6, and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a FFA releasing enzyme selected from the group consisting of FFA releasing enzymes comprising UniProt sequence #G0RGQ0, G0RH85, G0RMI3, G0RLH4, G0RIU1, G0RBM4, G0R9D1, G0RFR3, G0RG60, G0R6T6, G0R8N5, G0RBJ0, G0RRQ4, G0REZ4, G0RIJ9, G0R6X2, G0RJY0, G0RR42, G0RW77, G0RQJ5, G0RFT3, G0R810, G0RI29, G0RL87, G0RLL0, G0RGD5, and G0RKH7, and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a FFA releasing enzyme selected from the group consisting of FFA releasing enzymes comprising UniProt sequence #G0RH85, G0RGQ0, G0RLH4, and G0RMI3, and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a FFA releasing enzyme selected from the group consisting of FFA releasing enzymes comprising UniProt sequence #G0RH85 and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a FFA releasing enzyme selected from the group consisting of FFA releasing enzymes comprising UniProt sequence #G0RGQ0 and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a FFA releasing enzyme selected from the group consisting of FFA releasing enzymes comprising UniProt sequence #G0RLH4 and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a FFA releasing enzyme selected from the group consisting of FFA releasing enzymes comprising UniProt sequence #G0RMI3 and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, R0RVD2, and G0R8A6, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0, G0RH85, G0RMI3, G0RLH4, G0RIU1, G0RBM4, G0R9D1, G0RFR3, G0RG60, G0R6T6, G0R8N5, G0RBJ0, G0RRQ4, G0REZ4, G0RIJ9, G0R6X2, G0RJY0, G0RR42, G0RW77, G0RQJ5, G0RFT3, G0R810, G0RI29, G0RL87, G0RLL0, G0RGD5, and G0RKH7, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein and the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0, G0RLH4, G0RMI3, G0R707, G0R7K1, G0R810, G0RFT3, G0RG60, G0RGD5, G0RI29, G0RIJ9, G0RKH7, G0RKL4, G0RL87, G0RLL0, G0RLR3, G0RME5, G0RQJ5, G0RRK3, G0RSK7, G0RWT9, and G0RX82, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0, G0RLH4, and G0RMI3, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0, G0RLH4, G0RMI3, G0R707, G0R7K1, G0R810, G0RFT3, G0RG60, G0RGD5, G0RI29, G0RIJ9, G0RKH7, G0RKL4, G0RL87, G0RLL0, G0RLR3, G0RME5, G0RQJ5, G0RRK3, G0RSK7, G0RWT9, and G0RX82, and homologs thereof, and combinations thereof; and comprise an un-modulated production and/or activity of a third FFA releasing enzyme selected from the group consisting of FFA releasing enzymes comprising UniProt sequence #G0R6T6, G0R6X2, G0R8N5, G0R9D1, G0R9X3, G0RBJ0, G0RBM4, G0REM9, G0REZ4, G0RFR3, G0RHJ4, G0RIU1, G0RJY0, G0RR42, G0RV93, G0RRQ4, G0RX90, and G0RW77, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RIU1 and homologs thereof, and combinations thereof; and a modulated production and/or activity of a third FFA releasing enzyme selected from the group consisting of FFA releasing enzymes comprising UniProt sequence #G0RHJ4 and homologs thereof, and combinations thereof; and a modulated production and/or activity of a fourth FFA releasing enzyme selected from the group consisting of FFA releasing enzymes comprising UniProt sequence #G0RR42, G0RQJ5, G0RQD1, G0REZ4, G0RSK7, G0RV93, G0R9D1, G0R6T6, G0RLL0, G0RGD5, G0RFR3, G0RHQ7, G0RG60, G0R810, G0RL87, G0RIJ9, G0RME5, G0RRQ4, G0RX82, G0RGQ0, G0RLR3, G0RLH4, G0RFT3, G0RWY5, G0R9F9, G0RVD2, G0R8A6, G0R9X3, G0RBM4, G0RHJ4, G0REM9, G0RIU1, G0RX90, G0R8N5, G0R6X2, G0RK83, G0RKH7, G0RI29, G0RKI9, G0RJ76, G0RBJ0, G0RJY0, G0RMI3, G0RW77, G0RRK3, and G0RLB0, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; and a modulated production and/or activity of a third FFA releasing enzyme selected from the group consisting of FFA releasing enzymes comprising UniProt sequence #G0RGQ0 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; and a modulated production and/or activity of a third FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0 and homologs thereof, and combinations thereof; and a modulated production and/or activity of a third FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; and a modulated production and/or activity of a third FFA releasing enzyme selected from the group consisting of FFA releasing enzymes comprising UniProt sequence #G0RGQ0 and homologs thereof, and combinations thereof; and a modulated production and/or activity of a fourth FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the fourth FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

A modulated FFA releasing enzyme activity according to any of the above may comprise or consist of a modulated production and/or activity of a FFA releasing enzyme selected from the group consisting of FFA releasing enzymes comprising UniProt sequence #G0RH85, G0RGQ0, G0RLH4, and G0RMI3, and homologs thereof, and combinations thereof; and a modulated production and/or activity of a transcription factor selected from the group consisting of transcription factors comprising UniProt sequence #G0RT83, G0RRR1, G0RIF9, G0R765, G0RRJ7, G0R932, G0RBV8, G0REE8, G0RLF0, and G0RBH2, and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the transcription factor is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

### Recombinant Host Cell Comprising Modulated FFA Releasing Enzyme Activity

The method according to any of the above may comprise: i) obtaining a recombinant host cell capable of producing a recombinant component, wherein the recombinant host cell comprises a modulated FFA releasing enzyme activity compared to the FFA releasing enzyme activity comprised in a corresponding recombinant host cell (i.e., a recombinant host cell that is identical to the recombinant host cell that is being compared to the “corresponding recombinant host cell” except that the FFA releasing enzyme activity of the “corresponding recombinant host cell” is not modulated); and ii) culturing the recombinant host cell in a culture medium under conditions suitable for production and/or secretion of the recombinant component.

Accordingly, in various aspects, provided herein is a recombinant host cell that is capable of producing a recombinant component (i.e., that comprises an expression constructed provided herein) and that comprises a modulated FFA releasing enzyme activity (e.g., activity of any one FFA releasing enzyme disclosed herein or activities of any combination of two or more FFA releasing enzymes disclosed herein) compared to the FFA releasing enzyme activity comprised in a corresponding recombinant host cell.

A FFA releasing enzyme activity comprised in a recombinant host cell may be modulated by any means. For example, a FFA releasing enzyme activity may be modulated by a genetic modification in the recombinant host cell that modulates or essentially eliminates expression (i.e., production of an active protein) of the FFA releasing enzyme, or that modulates or essentially eliminates activity of the FFA releasing enzyme. Non-limiting examples of genetic modifications that may be comprised in a recombinant host cell according to any of the above include: i) a genetic modification in a polynucleotide sequence that encodes an endogenous FFA releasing enzyme or a functional part thereof (e.g., a catalytic domain), wherein the genetic modification modulates or essentially eliminates activity of the endogenous FFA releasing enzyme; ii) a genetic modification that introduces a polynucleotide sequence that encodes a heterologous FFA releasing enzyme, wherein the genetic modification modulates or essentially eliminates activity of the heterologous FFA releasing enzyme; iii) a genetic modification in a regulatory element or a functional part thereof (i.e., a part that is sufficient for the function of the regulatory element) that drives expression of a FFA releasing enzyme, wherein the genetic modification modulates or essentially eliminates expression of the FFA releasing enzyme; iv) a genetic modification in a coding sequence that encodes a protein required for expression of a FFA releasing enzyme (e.g., a transcription factor [e.g., any of the transcription factors shown in Table 3, and homologs thereof], a post-translational modification enzyme required for production of an active form of a FFA releasing enzyme) or for activity of a FFA releasing enzyme (e.g., an inhibitor, an activator, a co-enzyme), or a functional part thereof (e.g., DNA binding domain of a transcription factor, catalytic domain of a post-translational modification enzyme), wherein the genetic modification modulates or essentially eliminates activity of the protein required for expression or activity of the FFA releasing enzyme and thereby modulates or essentially eliminates expression of the FFA releasing enzyme; v) a genetic modification in a regulatory element or a functional part thereof that drives expression of a protein required for expression or activity of a FFA releasing enzyme, wherein the genetic modification modulates or essentially eliminates expression of the protein required for expression or activity of the FFA releasing enzyme and thereby modulates or essentially eliminates expression of the FFA releasing enzyme; and/or vi) a genetic modification that introduces a polynucleotide sequence that encodes a heterologous protein that regulates expression of a FFA releasing enzyme (e.g., a transcription factor [e.g., any of the transcription factors shown in Table 3, and homologs thereof], a post-translational modification enzyme required for production of an active form of a FFA releasing enzyme) or activity of a FFA releasing enzyme (e.g., an inhibitor, an activator, a co-enzyme) wherein the genetic modification provides for production of the heterologous protein and thereby modulates or essentially eliminates activity of the FFA releasing enzyme.

TABLE 3
Exemplary Transcription Factors (UniProt Sequence IDs)

| | | | |
|---|---|---|---|
| G0RX49 | G0RRJ7 | A2R2J1 | G2Q2Z5 |
| G0RHG1 | G0R932 | A2R903 | G2Q8I6 |
| G0RBV8 | G0REE8 | G0RLF0 | G0RBH2 |
| G0RT83 | G0RIF9 | G0RRR1 | G0R765 |

The recombinant host cell according to any of the above may be derived from any wild type unicellular organism, including any bacterium, yeast, filamentous fungus, archaea, unicellular protista, unicellular animals, unicellular plants, unicellular algae, protozoa, and chromista, or from a genetic variant (e.g., mutant) thereof, as well as from any generally recognized as safe (GRAS) industrial host cell.

Non-limiting examples of suitable bacteria include firmicutes, cyanobacteria (blue-green algae), oscillatoriophcideae, bacillales, lactobacillales, oscillatoriales, bacillaceae, lactobacillaceae, and members of any of the following genera, and derivatives and crosses thereof: Acinetobacter, Acetobacter (e.g., Acetobacter suboxydans, Acetobacter xylinum), Actinoplane (e.g., Actinoplane missouriensis), Arthrospira (e.g., Arthrospira platensis, Arthrospira maxima), Bacillus (e.g., Bacillus cereus, Bacillus coagulans, Bacillus licheniformis, Bacillus stearothermophilus, Bacillus subtilis), Escherichia (e.g., Escherichia coli), Lactobacillus (e.g., Lactobacillus acidophilus, Lactobacillus bulgaricus), Lactococcus (e.g., Lactococcus lactis, Lactococcus lactis Lancefield Group N, Lactobacillus reuteri), Leuconostoc (e.g., Leuconostoc citrovorum, Leuconostoc dextranicum, Leuconostoc mesenteroides), Micrococcus (e.g., Micrococcus lysodeikticus), Rhodococcus (e.g., Rhodococcus opacus, Rhodococcus opacus strain PD630), Spirulina, Streptococcus (e.g., Streptococcus cremoris, Streptococcus lactis, Streptococcus lactis subspecies diacetylactis, Streptococcus thermophilus), Streptomyces (e.g., Streptomyces chattanoogensis, Streptomyces griseus, Streptomyces natalensis, Streptomyces olivaceus, Streptomyces olivochromogenes, Streptomyces rubiginosus), Tetrahymena (e.g., Tetrahymena thermophile, Tetrahymena hegewischi, Tetrahymena hyperangularis, Tetrahymena malaccensis, Tetrahymena pigmentosa, Tetrahymena pyriformis, Tetrahymena vorax), and Xanthomonas (e.g., Xanthomonas campestris).

Non-limiting examples of suitable yeast include members of any of the following genera, and derivatives and crosses thereof: Candida (e.g., Candida albicans, Candida etchellsii, Candida guilliermondii, Candida humilis, Candida lipolytica, Candida orthopsilosis, Candida palmioleophila, Candida pseudotropicalis, Candida sp., Candida utilis, Candida versatilis), Cladosporium, Cryptococcus (e.g., Cryptococcus terricolus, Cryptococcus curvatus), Debaryomyces (e.g., Debaryomyces hansenii), Endomyces (e.g., Endomyces vernalis), Endomycopsis (e.g., Endomycopsis vernalis), Eremothecium (e.g., Eremothecium ashbyii), Hansenula (e.g., Hansenula sp., Hansenula polymorpha), Kluyveromyces (e.g., Kluyveromyces sp., Kluyveromyces lactis, Kluyveromyces marxianus var. lactis, Kluyveromyces marxianus, Kluyveromyces thermotolerans), Lipomyces (e.g., Lipomyces starkeyi, Lipomyecs lipofer), Ogataea (e.g., Ogataea minuta), Pichia (e.g., Pichia sp., Pichia pastoris, Pichia finlandica, Pichia trehalophila, Pichia koclamae, Pichia membranaefaciens, Pichia minuta, Pichia lindneri), Pichia opuntiae, Pichia thermotolerans, Pichia salictaria, Pichia guercuum, Pichia pijperi, Pichia stiptis, Pichia methanolica), Rhodosporidium (e.g., Rhodosporidium toruloides), Rhodotorula (e.g., Rhodotorula sp., Rhodotorula gracilis, Rhodotorula glutinis, Rhodotorula graminis), Saccharomyces (e.g., Saccharomyces sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum), Sporobolomyces (e.g., Sporobolomyces roseus), Sporidiobolus (e.g., Sporidiobolus johnsonii, Sporidiobolus salmonicolor), Trichosporon (e.g., Trichosporon cacaoliposimilis, Trichosporon oleaginosus sp. nov., Trichosporon cacaoliposimilis sp. nov., Trichosporon gracile, Trichosporon dulcitum, Trichosporon jirovecii, Trichosporon insectorum), Xanthophyllomyces (e.g., Xanthophyllomyces dendrorhous), Yarrowia (e.g., Yarrowia lipolytica), and Zygosaccharomyces (e.g., Zygosaccharomyces rouxii).

Non-limiting examples of suitable filamentous fungi include holomorphic, teleomorphic, and anamorphic forms of fungi, including members of any of the following genera, and derivatives and crosses thereof: Acremonium (e.g., Acremonium alabamense), Aspergillus (e.g., Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus niger var. awamori, Aspergillus ochraceus, Aspergillus oryzae, Aspergillus sojae, Aspergillus terreus, as well as Emericella, Neosartorya, and Petromyces species), Aureobasidium, Canariomyces, Chaetomium, Chaetomidium, Corynascus, Chrysosporium (e.g., Chrysosporium botryoides, Chrysosporium carmichaeli, Chrysosporium crassitunicatum, Chrysosporium europae, Chrysosporium evolceannui, Chrysosporium farinicola, Chrysosporium fastidium, Chrysosporium filiforme, Chrysosporium georgiae, Chrysosporium globiferum, Chrysosporium globiferum var. articulatum, Chrysosporium globiferum var. niveum, Chrysosporium hirundo, Chrysosporium hispanicum, Chrysosporium holmii, Chrysosporium indicum, Chrysosporium iops, Chrysosporium keratinophilum, Chrysosporium kreiselii, Chrysosporium kuzurovianum, Chrysosporium lignorum, Chrysosporium obatum, Chrysosporium lucknowense, Chrysosporium lucknowense Garg 27K, Chrysosporium medium, Chrysosporium medium var. spissescens, Chrysosporium mephiticum, Chrysosporium merdarium, Chrysosporium merdarium var. roseum, Chrysosporium minor, Chrysosporium pannicola, Chrysosporium parvum, Chrysosporium parvum var. crescens, Chrysosporium pilosum, Chrysosporium pseudomerdarium, Chrysosporium pyriformis, Chrysosporium queenslandicum, Chrysosporium sigleri, Chrysosporium sulfureum, Chrysosporium synchronum, Chrysosporium tropicum, Chrysosporium undulatum, Chrysosporium vallenarense, Chrysosporium vespertilium, Chrysosporium zonatum), Coonemeria, Cunninghamella (e.g., Cunninghamella ehinulata), Dactylomyces, Emericella, Filibasidium, Fusarium (e.g., Fusarium moniliforme, Fusarium venenatum, Fusarium oxysporum, Fusarium graminearum, Fusarium proliferatum, Fusarium verticiollioides, Fusarium culmorum, Fusarium crookwellense, Fusarium poae, Fusarium sporotrichioides, Fusarium sambuccinum, Fusarium torulosum, as well as associated Gibberella teleomorphic forms thereof), Gibberella, Humicola, Hypocrea, Lentinula, Malbranchea (e.g., Malbranchea filamentosa), Magnaporthe, Malbranchium, Melanocarpus, Mortierella (e.g., Mortierella alpina 1S-4, Mortieralla isabelline, Mortierrla vinacea, Mortieralla vinaceae var. raffinoseutilizer), Mucor (e.g., Mucor miehei Cooney et Emerson (Rhizomucor miehei (Cooney & R. Emerson)) Schipper, Mucor pusillus Lindt, Mucor circinelloides Mucor mucedo), Myceliophthora (e.g., Myceliophthora thermophila), Myrothecium, Neocallimastix, Neurospora (e.g., Neurospora crassa), Paecilomyces, Penicillium (e.g., Penicillium chrysogenum, Pennicillium iilacinum, Penicillium roquefortii), Phenerochaete, Phlebia, Piromyces, Pythium, Rhizopus (e.g., Rhizopus niveus), Schizophyllum, Scytalidium, Sporotrichum (e.g., Sporotrichum cellulophilum), Stereum, Talaromyces, Thermoascus, Thermomyces, Thielavia (e.g., Thielavia terrestris), Tolypocladium, and Trichoderma (e.g., Trichoderma harzianum, Trichoderma koningii, Trichoderma longibrachiatum, Trichoderma reesei, Trichoderma atroviride, Trichoderma virens, Trichoderma citrinoviride, Trichoderma viride).

The recombinant host cell according to any of the above may be selected from the group consisting of: a recombinant Trichoderma reesei host cell (i.e., a recombinant host cell derived from a Trichoderma reesei strain) comprising a modulated activity of a FFA releasing enzyme or of a combination of two or more FFA releasing enzymes, a recombinant Aspergillus niger host cell (i.e., a recombinant host cell derived from a Aspergillus niger strain) comprising a modulated activity of a FFA releasing enzyme or of a combination of two or more FFA releasing enzymes, a recombinant Trichoderma citrinoviride host cell (i.e., a recombinant host cell derived from a Trichoderma citrinoviride strain) comprising a modulated activity of a FFA releasing enzyme or of a combination of two or more FFA releasing enzymes, and a recombinant Myceliophthora thermophila host cell (i.e., a recombinant host cell derived from a Myceliophthora thermophila strain) comprising a modulated activity of a FFA releasing enzyme or of a combination of two or more FFA releasing enzymes.

The recombinant host cell according to any of the above may comprise a modulated production and/or activity of a FFA releasing enzyme selected from the group consisting of FFA releasing enzymes comprising UniProt sequence #G0RH85, G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, G0RVD2, and G0R8A6, and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a FFA releasing enzyme selected from the group consisting of FFA releasing enzymes comprising UniProt sequence #G0RGQ0, G0RH85, G0RMI3, G0RLH4, G0RIU1, G0RBM4, G0R9D1, G0RFR3, G0RG60, G0R6T6, G0R8N5, G0RBJ0, G0RRQ4, G0REZ4, G0RIJ9, G0R6X2, G0RJY0, G0RR42, G0RW77, G0RQJ5, G0RFT3, G0R810, G0RI29, G0RL87, G0RLL0, G0RGD5, and G0RKH7, and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a FFA releasing enzyme selected from the group consisting of FFA releasing enzymes comprising UniProt sequence #G0RH85, G0RGQ0, G0RLH4, and G0RMI3, and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a FFA releasing enzyme selected from the group consisting of FFA releasing enzymes comprising UniProt sequence #G0RH85 and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a FFA releasing enzyme selected from the group consisting of FFA releasing enzymes comprising UniProt sequence #G0RGQ0 and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a FFA releasing enzyme selected from the group consisting of FFA releasing enzymes comprising UniProt sequence #G0RLH4 and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a FFA releasing enzyme selected from the group consisting of FFA releasing enzymes comprising UniProt sequence #G0RMI3 and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, R0RVD2, and G0R8A6, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0, G0RH85, G0RMI3, G0RLH4, G0RIU1, G0RBM4, G0R9D1, G0RFR3, G0RG60, G0R6T6, G0R8N5, G0RBJ0, G0RRQ4, G0REZ4, G0RIJ9, G0R6X2, G0RJY0, G0RR42, G0RW77, G0RQJ5, G0RFT3, G0R810, G0RI29, G0RL87, G0RLL0, G0RGD5, and G0RKH7, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein and the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0, G0RLH4, G0RMI3, G0R707, G0R7K1, G0R810, G0RFT3, G0RG60, G0RGD5, G0RI29, G0RIJ9, G0RKH7, G0RKL4, G0RL87, G0RLL0, G0RLR3, G0RME5, G0RQJ5, G0RRK3, G0RSK7, G0RWT9, and G0RX82, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0, G0RLH4, and G0RMI3, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0, G0RLH4, G0RMI3, G0R707, G0R7K1, G0R810, G0RFT3, G0RG60, G0RGD5, G0RI29, G0RIJ9, G0RKH7, G0RKL4, G0RL87, G0RLL0, G0RLR3, G0RME5, G0RQJ5, G0RRK3, G0RSK7, G0RWT9, and G0RX82, and homologs thereof, and combinations thereof; and comprise an un-modulated production and/or activity of a third FFA releasing enzyme selected from the group consisting of FFA releasing enzymes comprising UniProt sequence #G0R6T6, G0R6X2, G0R8N5, G0R9D1, G0R9X3, G0RBJ0, G0RBM4, G0REM9, G0REZ4, G0RFR3, G0RHJ4, G0RIU1, G0RJY0, G0RR42, G0RV93, G0RRQ4, G0RX90, and G0RW77, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RIU1 and homologs thereof, and combinations thereof; and a modulated production and/or activity of a third FFA releasing enzyme selected from the group consisting of FFA releasing enzymes comprising UniProt sequence #G0RHJ4 and homologs thereof, and combinations thereof; and a modulated production and/or activity of a fourth FFA releasing enzyme selected from the group consisting of FFA releasing enzymes comprising UniProt sequence #G0RR42, G0RQJ5, G0RQD1, G0REZ4, G0RSK7, G0RV93, G0R9D1, G0R6T6, G0RLL0, G0RGD5, G0RFR3, G0RHQ7, G0RG60, G0R810, G0RL87, G0RIJ9, G0RME5, G0RRQ4, G0RX82, G0RGQ0, G0RLR3, G0RLH4, G0RFT3, G0RWY5, G0R9F9, G0RVD2, G0R8A6, G0R9X3, G0RBM4, G0RHJ4, G0REM9, G0RIU1, G0RX90, G0R8N5, G0R6X2, G0RK83, G0RKH7, G0RI29, G0RKI9, G0RJ76, G0RBJ0, G0RJY0, G0RMI3, G0RW77, G0RRK3, and G0RLB0, and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; and a modulated production and/or activity of a third FFA releasing enzyme selected from the group consisting of FFA releasing enzymes comprising UniProt sequence #G0RGQ0 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; and a modulated production and/or activity of a third FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RGQ0 and homologs thereof, and combinations thereof; and a modulated production and/or activity of a third FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RH85 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; and a modulated production and/or activity of a third FFA releasing enzyme selected from the group consisting of FFA releasing enzymes comprising UniProt sequence #G0RGQ0 and homologs thereof, and combinations thereof; and a modulated production and/or activity of a fourth FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the fourth FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a FFA releasing enzyme selected from the group consisting of FFA releasing enzymes comprising UniProt sequence #G0RH85, G0RGQ0, G0RLH4, and G0RMI3, and homologs thereof, and combinations thereof; and a modulated production and/or activity of a transcription factor selected from the group consisting of transcription factors comprising UniProt sequence #G0RT83, G0RRR1, G0RIF9, G0R765, G0RRJ7, G0R932, G0RBV8, G0REE8, G0RLF0, and G0RBH2, and homologs thereof, and combinations thereof; wherein the modulated production and/or activity of the FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the transcription factor is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RGQ0 and homologs thereof, and combinations thereof; and a modulated production and/or activity of a second FFA releasing enzyme selected from the group consisting of FFA releasing enzymes comprising UniProt sequence #G0RLH4 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of a first FFA releasing enzyme selected from the group consisting of FFA releasing enzymes comprising UniProt sequence #G0RGQ0 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RLH4 and homologs thereof, and combinations thereof; and a modulated production and/or activity of a second FFA releasing enzyme selected from the group consisting of FFA releasing enzymes comprising UniProt sequence #G0RMI3 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

The recombinant host cell according to any of the above may comprise a modulated production and/or activity of a first FFA releasing enzyme selected from the group consisting of FFA releasing enzymes comprising UniProt sequence #G0RGQ0 and homologs thereof, and combinations thereof; and a modulated production and/or activity of a second FFA releasing enzyme selected from the group consisting of FFA releasing enzymes comprising UniProt sequence #G0RLH4 and homologs thereof, and combinations thereof; and a modulated production and/or activity of a third FFA releasing enzyme selected from the group consisting of FFA releasing enzymes comprising UniProt sequence #G0RMI3 and homologs thereof, and combinations thereof; wherein the modulated production and/or activity of the first FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; wherein the modulated production and/or activity of the second FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; and wherein the modulated production and/or activity of the third FFA releasing enzyme is a decrease in production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

### Method for Obtaining Recombinant Host Cell

In various aspects, provided herein is a method for obtaining a recombinant host cell according to any of the above, wherein the method comprises any combination, in any order, of: i) obtaining a polynucleotide according to any of the below, or a recombinant expression construct according to any of the below, or a recombinant vector according to any of the below; ii) introducing the polynucleotide, expression construct, or recombinant vector into a host cell (e.g., any of the host cells disclosed herein) to obtain a recombinant host cell capable of producing a recombinant component; iii) genetically modifying a host cell (e.g., any of the host cells disclosed herein) to modulate or essentially eliminate production and/or activity of a FFA releasing enzyme (e.g., any one of the FFA releasing enzymes disclosed herein or any combination of two or more FFA releasing enzymes disclosed herein) to obtain a recombinant host cell comprising a modified or essentially eliminated FFA releasing enzyme activity; iv) introducing the polynucleotide, expression construct, or recombinant vector into a recombinant host cell comprising a modified or essentially eliminated FFA releasing enzyme activity; and/or v) genetically modifying a recombinant host cell capable of producing a recombinant component to modulate or essentially eliminate production and/or activity of a FFA releasing enzyme (e.g., any one of the FFA releasing enzymes disclosed herein or any combination of two or more FFA releasing enzymes disclosed herein).

### Polynucleotide

The polynucleotide, expression construct, and/or recombinant vector may be obtained by any suitable method known in the art, including, without limitation, direct chemical synthesis and cloning.

The polynucleotide may comprise: i) an optional secretion signal sequence (i.e., a sequence that encodes a peptide that mediates the delivery of a nascent protein attached to the peptide to the exterior of the cell in which the nascent protein is synthesized; e.g., a polynucleotide sequence encoding any of the secretion signals disclosed herein), and ii) a recombinant protein coding sequence (i.e., a polynucleotide sequence encoding a recombinant protein (e.g., any of the recombinant proteins provided herein), optionally comprising a tag polypeptide (e.g., any of the tag polypeptides disclosed herein)), wherein: a) the optional secretion signal sequence is operably linked in sense orientation to the recombinant protein coding sequence (i.e., the optional secretion signal sequence and the recombinant protein coding sequence are positioned such that transcription and translation produces a recombinant protein comprising the optional secretion signal).

### Secretion Signal Sequence

The recombinant expression construct according to any of the above may optionally comprise any secretion signal sequence that is active in a recombinant host cell according to any of the below.

The optional secretion signal sequence may encode a secretion signal that mediates translocation of the nascent recombinant protein into the ER post-translationally (i.e., protein synthesis precedes translocation such that the nascent recombinant protein is present in the cell cytosol prior to translocating into the ER) or co-translationally (i.e., protein synthesis and translocation into the ER occur simultaneously).

Non-limiting examples of suitable secretion signal sequences include secretion signal sequences that are functional in a bacterial host cell, including secretion signal sequences of genes encoding any of the following proteins: PelB, OmpA, Bla, PhoA, PhoS, MalE, LivK, LivJ, MglB, AraF, AmpC, RbsB, MerP, CpdB, Lpp, LamB, OmpC, PhoE, OmpF, TolC, BtuB, and LutA, and functional parts and combinations thereof.

Non-limiting examples of suitable secretion signal sequences include secretion signal sequences that are functional in a fungal host cell, including secretion signal sequences of genes encoding any of the following proteins: CBH1, CBH2, EGL1, EGL2, XYN1, XYN2, BXL1, HFB1, HFB2, GLAA, AMYA, AMYC, AAMA, alpha mating factor, SUC2, PHO5, INV, AMY, LIP, PIR, OST1, and β-glucosidase, and functional parts and combinations thereof.

### Recombinant Protein Coding Sequence

The recombinant protein coding sequence may encode any recombinant protein.

Non-limiting examples of recombinant proteins include milk proteins.

The term “milk protein” as used herein refers to a whey protein or a casein.

The term “casein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a casein natively found in a mammal-produced milk (i.e., a casein that is native to a milk produced by a mammal; e.g., a native casein). Examples of caseins include β-casein, κ-casein, α-S1-casein, and α-S2-casein. Accordingly, the terms “β-casein”, “κ-casein”, “α-S1-casein”, and “α-S2-casein” as used herein refer to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a β-casein, κ-casein, α-S1-casein, and α-S2-casein, respectively, natively found in a mammal-produced milk (e.g., Bos taurus β-casein (amino acids 16 to 224 of UniProt sequence P02666), Bos taurus κ-casein (amino acids 22 to 190 of UniProt sequence P02668), Bos taurus α-S1-casein (amino acids 16 to 214 of UniProt sequence P02662), and Bos taurus α-S2-casein (amino acids 16 to 222 of UniProt sequence P02663), respectively).

The term “whey protein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a whey protein natively found in a mammal-produced milk (i.e., a whey protein that is native to a milk produced by a mammal; e.g., a native whey protein). Examples of whey proteins include α-lactalbumin, β-lactoglobulin, lactotransferrin, lactoferricin, serum albumin protein, lactoperoxidase protein, and glycomacropeptide. Accordingly, the terms “α-lactalbumin”, “β-lactoglobulin”, “lactotransferrin”, “lactoferricin”, “serum albumin”, “lactoperoxidase”, and “glycomacropeptide” as used herein refer to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in an α-lactalbumin, β-lactoglobulin, lactotransferrin, lactoferricin, serum albumin, lactoperoxidase, and glycomacropeptide (GMP), respectively, natively found in a mammal-produced milk (e.g., Bos taurus α-lactalbumin (amino acids 20-142 of UniProt sequence P00711), Bos taurus β-lactoglobulin (amino acids 17-178 of UniProt sequence P02754), Bos taurus lactotransferrin (amino acids 20 to 708 of UniProt sequence P24627), Bos taurus lactoferricin (amino acids 36 to 60 of UniProt sequence P24627), Bos taurus serum albumin (amino acids 25 to 607 of UniProt sequence P02769), Bos taurus lactoperoxidase (amino acids 101 to 712 of UniProt sequence P80025), and Bos taurus glycomacropeptide (GMP; amino acids 127 to 190 of UniProt sequence P02668), respectively).

The milk protein may have an amino acid sequence that is identical to or a homolog of an amino acid sequence of a native milk protein found in any mammalian species, including but not limited to cow, human, sheep, mouflon, goat, buffalo, camel, horse, donkey, alpaca, yak, llama, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, and echidna.

### Recombinant Expression Construct

The recombinant expression construct may comprise: i) a promoter sequence (e.g., a polynucleotide sequence for any of the promoters disclosed herein), ii) the polynucleotide of any of the above, and iii) a termination sequence (e.g., a polynucleotide sequence for any of the terminators disclosed herein); wherein: a) the promoter sequence is operably linked in sense orientation to the optional secretion signal sequence and the recombinant protein coding sequence of the polynucleotide (i.e., the promoter sequence and the optional secretion signal sequence and the recombinant protein coding sequence of the polynucleated are positioned such that the promoter sequence is effective in mediating or regulating transcription of the optional secretion signal sequence and the recombinant protein coding sequence), and b) the one or more terminator sequences are operably linked to the recombinant protein coding sequence (i.e., the recombinant protein coding sequence and the one or more terminator sequences are positioned such that the one or more terminator sequences are effective in terminating transcription of the recombinant protein coding sequence).

The recombinant expression construct may further comprise an operably linked sequence encoding for an affinity purification tag, such that the expressed recombinant protein includes a peptide sequence for affinity purification. Such affinity purification tag may be operably linked such that when expressed the affinity purification tag is present either at or toward the amino terminus, the carboxy terminus, or both. Such affinity purification tag may be a maltose binding protein (MBP) tag, a glutathione-S-transferase (GST) tag, a poly(His) tag, a hexa(His) tag, a FLAG-tag, a V5-tag, a VSV-tag, an E-tag, an NE-tag, a hemagglutinin (Ha)-tag, and a Myc-tag.

The recombinant expression construct may further comprise a sequence for integration by homologous (i.e., targeted integration) or nonhomologous recombination into the genome of a host cell. The recombinant expression construct may comprise at least 10, at least 25, at least 50, at least 100, at least 250, at least 500, at least 750, at least 1,000, or at least 10,000 base pairs that have sufficient identity with a target sequence in the genome of the host cell to enhance the probability of homologous recombination of the recombinant expression construct. Such homologous sequence may be non-coding or coding.

The optional secretion signal sequence and/or recombinant protein coding sequence comprised in the recombinant expression construct according to any of the above may be codon-optimized for expression in the recombinant host cell according to any of the above.

The recombinant expression construct according to any of the above may be generated upon integration of a fragment of the recombinant expression construct into the genome of a host cell (e.g., the genome of the recombinant host cell according to any of the above). For example, a polynucleotide according to any of the above may be stably integrated within the genome of a host cell such that one or more regulatory elements of an endogenous gene locus become operably linked to the recombinant protein coding sequence, thereby generating the recombinant expression construct according to any of the above.

### Promoter Sequence

The recombinant expression construct according to any of the above may comprise any promoter sequence that is active in a recombinant host cell according to any of the below.

The promoter sequence may be a constitutive promoter sequence (i.e., a promoter sequence that is active under most environmental and developmental conditions), or an inducible or repressible promoter sequence (i.e., a promoter sequence that is active only under certain environmental or developmental conditions [e.g., in presence or absence of certain factors, such as, but not limited to, carbon (e.g., glucose, galactose, lactose, sucrose, cellulose, sophorose, gentiobiose, sorbose, disaccharides that induce the cellulase promoters, starch, tryptophan, thiamine, methanol), phosphate, nitrogen, or other nutrient; temperature; pH; osmolarity; heavy metals or heavy metal ions; inhibitors; stress; catabolites; and combinations thereof]).

The promoter sequence may consist of a single promoter sequence, or of two or more promoter sequences (e.g., combination of two or more promoters or functional parts thereof arranged in sequence, combination of an inducible and a constitutive promoter). The two or more promoter sequences may be identical, or at least two of the two or more promoter sequences cannot be identical.

The promoter sequence may comprise or consist of a bidirectional promoter sequence (i.e., a polynucleotide that initiates transcription in both orientations by recruiting transcription factors, for example generated by fusing two identical or different promoters in opposite directions).

Non-limiting examples of suitable promoter sequences include promoter sequences that are functional in a bacterial host cell, including T7 promoter, T5 promoter, Tac promoter, pL/pR promoter, phoA promoter, lacUV5 promoter, trc promoter, trp promoter, cstA promoter, xylA promoter, manP promoter, malA promoter, lacA promoter, aprE promoter, ΔaprE promoter, srfA promoter, p43 promoter, ylbA promoter, GB promoter, veg promoter, PG1 promoter, PG6 promoter, λPL promoter, λPR promoter, and spa promoter, and functional parts and combinations thereof.

Non-limiting examples of suitable promoter sequences include promoter sequences that are functional in a fungal host cell, including xlnA promoter, xyn1 promoter, xyn2 promoter, xyn3 promoter, xyn4 promoter, bx11 promoter, cbh1 promoter, cbh2 promoter, eg11 promoter, eg12 promoter, eg13 promoter, eg14 promoter, eg15 promoter, glaA promoter, agdA promoter, gpdA promoter, gpd1 promoter, AOX1 promoter, GAP1 promoter, MET3 promoter, ENO1 promoter, GPD1 promoter, PDC1 promoter, TEF1 promoter, AXE1 promoter, CIP1 promoter, GH61 promoter, PKI1 promoter, RP2 promoter, ADH1 promoter, CUP1 promoter, GAL1 promoter, PGK1 promoter, YPT1 promoter, LAC4 promoter, LAC4-PB1 promoter, FLD1 promoter, MOX promoter, DAS1 promoter, DAS2 promoter, GAP1 promoter, STR3 promoter, ADH3 promoter, GUT2 promoter, CYC1 promoter, TDH3 promoter, PGL1 promoter, ADH2 promoter, HXT7 promoter, CLB1 promoter, and PHO5 promoter, and functional parts and combinations thereof.

### Termination Sequence

The recombinant expression construct according to any of the above may comprise any termination sequence that is active in a recombinant host cell according to any of the below.

Non-limiting examples of suitable termination sequences include termination sequences of adh1, amaA, amdS, amyA, aox1, cbh1, cbh2, cyc2, eg11, eg12, ga11, gap1, glaA, gpd1, gpdA, pdc1, pgk1, tef1, tps1, trpC, xyn1, xyn2, xyn3, and xyn4 genes, and functional parts and combinations thereof.

The termination sequence may consist of a single termination sequence, or of two or more termination sequences, wherein the two or more termination sequences may be identical, or at least two of the two or more termination sequences may be not identical. The termination sequence may consist of a bidirectional termination sequence.

### Additional Regulatory Elements

The recombinant expression construct according to any of the above may further comprise additional regulatory elements.

Non-limiting examples of regulatory elements include promoter sequences, termination sequences, transcriptional start sequences, translational start sequences, translation stop sequences, enhancer sequences, activator sequences, response elements, protein recognition sites, inducible elements, protein binding sequences, 5′ and 3′ untranslated regions (e.g., a 3′ untranslated region comprising a poly-adenylation signal), upstream activation sequences (UAS), introns, operators (i.e., sequences of nucleic acids adjacent to a promoter that comprise a protein-binding domain where a repressor protein can bind and reduce or eliminate activity of the promoter), efficient RNA processing signals (e.g., splicing signals, polyadenylation signals), sequences that stabilize cytoplasmic mRNA, sequences that enhance translation efficiency (e.g., ribosome binding sites [e.g., Shine-Dalgarno sequences]), sequences that enhance protein stability, sequences that enhance protein secretion, and combinations thereof.

### Recombinant Vector

The recombinant vector may comprise a recombinant expression construct according to any of the above.

The recombinant vector may comprise a single recombinant expression construct according to any of the above, or two or more recombinant expression constructs according to any of the above, which may be identical or at least two of which may be not identical (e.g., differ from each other in a promoter sequence, a secretion signal, a protein coding sequence, a termination sequence, and/or an additional regulatory element). In embodiments in which the recombinant vector comprises two or more recombinant expression constructs, the two or more recombinant expression constructs may encode the same recombinant protein. In some such embodiments, the two or more recombinant expression constructs encoding the same recombinant protein differ from each other in a promoter sequence, secretion signal sequence, termination sequence, and/or additional regulatory element.

The recombinant vector may further comprise one or more other elements suitable for propagation of the recombinant vector in a recombinant host cell. Non-limiting examples of such other elements include origins of replication and selection markers. Origins of replication and selection markers are known in the art, and include bacterial and fungal origins of replication (e.g., AMA1, ANSI). Selection markers may be resistance genes (i.e., polynucleotides that encode proteins that enable host cells to detoxify an exogenously added compound [e.g., an antibiotic compound]), auxotrophic markers (i.e., polynucleotides that encode proteins that permit a host cell to synthesize an essential component (usually an amino acid) while grown in media that lacks that essential component), or color markers (i.e., genes that encode proteins that can produce a color). Non-limiting examples of suitable selection markers include amdS (acetamidase), argB (ornithine carbamoyltransferase), bar (phosphinothricin acetyltransferase), hph (hygromycin phosphotransferase), niaD (nitrate reductase), pyrG (orotidine 5′-phosphate decarboxylase), sC (sulfate adenyltransferase), trpC (anthranilate synthase), and ble (bleomycin-type antibiotic resistance), and derivatives thereof. The selection marker may comprise an alteration that decreases production of the selective marker, thus increasing the number of copies needed to permit a recombinant host cell comprising the recombinant vector to survive under selection. Selection may also be accomplished by co-transformation, wherein the transformation is carried out with a mixture of two vectors and the selection is made for one vector only.

The recombinant vector may further comprise sequences for integration by homologous (i.e., targeted integration) or nonhomologous recombination into the genome of a host cell. The recombinant expression construct may comprise at least 10, at least 25, at least 50, at least 100, at least 250, at least 500, at least 750, at least 1,000, or at least 10,000 base pairs that have sufficient identity with a target sequence in the genome of the host cell to enhance the probability of homologous recombination of the recombinant expression construct. Such homologous sequence may be non-coding or coding.

### Genetic Modification

A genetic modification may consist of, for example, an insertion, a substitution, a duplication, a rearrangement and/or a deletion of one or more nucleotides in a genome of a cell. A genetic modification may, for example, introduce a stop codon; remove a start codon; insert a frame-shift of the open reading frame; or create a point mutation, missense mutation, substitution mutation, deletion mutation, frameshift mutation, insertion mutation, duplication mutation, amplification mutation, translocation mutation, or inversion mutation.

Methods for genetically modifying a host cell are well known in the art, and include, without limitation, random mutagenesis and screening, site-directed mutagenesis, PCR mutagenesis, insertional mutagenesis, chemical mutagenesis (using, for example, hydroxylamine, N-methyl-N′-nitro-N-nitrosoguanidine (MNNG), N-methyl-N′-nitrosogaunidine (NTG), O-methyl hydroxylamine, nitrous acid, ethyl methane sulphonate (EMS), sodium bisulphite, formic acid, nucleotide analogues), irradiation (e.g., ultraviolet (UV) irradiation), deletion of coding or non-coding nucleotide sequences, homologous recombination, FLP/FRT recombination, gene disruption, CRISPR gene editing, and gene conversion. Such methods include introduction into a host cell of a recombinant polynucleotide that comprises a polynucleotide sequence that is complementary to a polynucleotide sequence encoding a protein of interest (e.g., a FFA releasing enzyme, a protein that drives expression and/or regulates activity of a FFA releasing enzyme), that encodes a RNAi construct that is specific to a protein of interest, or that encodes a heterologous inhibitor or activator of a FFA releasing enzyme.

The modulated production and/or activity of a FFA releasing enzyme in the recombinant host cell according to any of the above may be evaluated using any suitable method known in the art, such as assays that are carried out at the RNA level and, most suitable, at the protein level, or by use of functional bioassays that measure the production or activity of the secretion-related protein. Non-limiting examples of such assays include Northern blotting, dot blotting (DNA or RNA), RT-PCR (reverse transcriptase polymerase chain reaction), in situ hybridization, Southern blotting, enzyme activity assays, immunological assays (e.g., immunohistochemical staining, immunoassays, Western blotting, ELISA), and free thiol assays (e.g., for measuring production of protein comprising free cysteine residues). The production and/or activity of a FFA releasing enzyme in the recombinant host cell according to any of the above may be compared to that of a corresponding recombinant host cell (i.e., an identical recombinant host cell that is also capable of producing the recombinant component (i.e., comprises the same expression construct) but that does not comprise the genetic modification that modulates or essentially eliminates the FFA releasing enzyme activity) evaluated by the same method.

Introducing Polynucleotide, Recombinant Expression Construct, or Recombinant Vector into Host Cell

Methods for introducing a polynucleotide, recombinant expression construct, or recombinant vector into a host cell are well-known in the art. Non-limiting examples of such methods include calcium phosphate transfection, dendrimer transfection, liposome transfection (e.g., cationic liposome transfection), cationic polymer transfection, DEAE-dextran transfection, cell squeezing, sonoporation, optical transfection, protoplast fusion, protoplast transformation, impalefection, hyrodynamic delivery, gene gun, magnetofection, viral transduction, electroporation, and chemical transformation (e.g., using PEG).

Methods for identifying a recombinant host cell are well-known in the art, and include screening for expression of a drug resistance or auxotrophic marker encoded by the polynucleotide, recombinant expression construct, or recombinant vector that permits selection for or against growth of cells, or by other means (e.g., detection of light emitting peptide comprised in the polynucleotide, recombinant expression construct, or recombinant vector, molecular analysis of individual recombinant host cell colonies [e.g., by restriction enzyme mapping, PCR amplification, Southern analysis, or sequence analysis of isolated extrachromosomal vectors or chromosomal integration sites]).

Production of the recombinant protein by the recombinant host cell according to any of the above may be evaluated using any suitable method known in the art, such as assays that are carried out at the RNA level and, most suitable, at the protein level, or by use of functional bioassays that measure the production or activity of the recombinant protein. Non-limiting examples of such assays include Northern blotting, dot blotting (DNA or RNA), RT-PCR (reverse transcriptase polymerase chain reaction), RNA-Seq, in situ hybridization, Southern blotting, enzyme activity assays, immunological assays (e.g., immunohistochemical staining, immunoassays, Western blotting, ELISA), and free thiol assays (e.g., for measuring production of protein comprising free cysteine residues)

### Method for Producing a Recombinant Component

In various aspects, provided herein is a method for producing the recombinant component according to any of the above, wherein the method comprises: fermenting a recombinant host cell according to any of the above in a culture medium under conditions suitable for production of the recombinant component.

The method may further comprise: purifying the recombinant component from the fermentation broth to obtain a preparation comprising the recombinant component; and/or post-processing the recombinant component.

### Fermenting

Suitable conditions for producing the recombinant component are typically those under which the recombinant host cell according to any of the above can grow and/or remain viable, and produce the recombinant component.

Non-limiting examples of suitable conditions include a suitable culture medium (e.g., a culture medium having a suitable nutrient content [e.g., a suitable carbon content, a suitable nitrogen content, a suitable phosphorus content], a suitable supplement content, a suitable trace metal content, a suitable pH), a suitable temperature, a suitable feed rate, a suitable pressure, a suitable level of oxygenation, a suitable fermentation duration (i.e., volume of culture media comprising the recombinant host cells), a suitable fermentation volume (i.e., volume of culture media comprising the recombinant host cells), and a suitable fermentation vessel.

Suitable culture media include all culture media in which the recombinant host cell can grow and/or remain viable, and produce the recombinant component. Typically, the culture medium is an aqueous medium that comprises a carbon source, an assimilable nitrogen source (i.e., a nitrogen-containing compound capable of releasing nitrogen in a form suitable for metabolic utilization by the recombinant host cell), and a phosphate source.

Non-limiting examples of carbon sources include monosaccharides, disaccharides, polysaccharides, acetate, ethanol, methanol, glycerol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, maltodextrin, and combinations thereof.

Non-limiting examples of assimilable nitrogen sources include anhydrous ammonia, ammonium sulfate, ammonium hydroxide, ammonium nitrate, diammonium phosphate, monoammonium phosphate, ammonium pyrophosphate, ammonium chloride, sodium nitrate, urea, peptone, protein hydrolysates, corn steep liquor, corn steep solids, spent grain, spent grain extract, and yeast extract. Use of ammonia gas is convenient for large scale operations, and may be employed by bubbling through the aqueous ferment (fermentation medium) in suitable amounts. At the same time, such ammonia may also be employed to assist in pH control.

The culture medium may further comprise an inorganic salt, a mineral (e.g., magnesium, calcium, potassium, sodium; e.g., in suitable soluble assimilable ionic and combined forms), a metal or transition metal (e.g., copper, manganese, molybdenum, zinc, iron, boron, iodine; e.g., in suitable soluble assimilable form), a vitamin, and any other nutrient or functional ingredient (e.g., a protease [e.g., a plant-based protease] that can prevent degradation of the recombinant component, a protease inhibitor that can reduce the activity of a protease that can degrade the recombinant component, and/or a sacrificial protein that can siphon away protease activity, an anti-foaming agent, an anti-microbial agent, a surfactant, an emulsifying oil).

Suitable culture media are available from commercial suppliers or may be prepared according to published compositions (e.g., in catalogues of the American Type Culture Collection).

A suitable pH may be a pH of between about 2 and about 8 (e.g., a pH of between 2 and 8, 7.5, 7, 6.5, 6, 5.5, 5.4, 5.3, 5.2, 5.1, 5, 4.9, 4.8, 4.7, 4.6, 4.5, 4, 3.5, 3, or 2.5; between 2.5 and 8, 7.5, 7, 6.5, 6, 5.5, 5.4, 5.3, 5.2, 5.1, 5, 4.9, 4.8, 4.7, 4.6, 4.5, 4, 3.5, or 3; between 3 and 8, 7.5, 7, 6.5, 6, 5.5, 5.4, 5.3, 5.2, 5.1, 5, 4.9, 4.8, 4.7, 4.6, 4.5, 4, or 3.5; between 3.5 and 8, 7.5, 7, 6.5, 6, 5.5, 5.4, 5.3, 5.2, 5.1, 5, 4.9, 4.8, 4.7, 4.6, 4.5, or 4; between 4 and 8, 7.5, 7, 6.5, 6, 5.5, 5.4, 5.3, 5.2, 5.1, 5, 4.9, 4.8, 4.7, 4.6, or 4.5; between 4.5 and 8, 7.5, 7, 6.5, 6, 5.5, 5.4, 5.3, 5.2, 5.1, 5, 4.9, 4.8, 4.7, or 4.6; between 4.6 and 8, 7.5, 7, 6.5, 6, 5.5, 5.4, 5.3, 5.2, 5.1, 5, 4.9, 4.8, or 4.7; between 4.7 and 8, 7.5, 7, 6.5, 6, 5.5, 5.4, 5.3, 5.2, 5.1, 5, 4.9, or 4.8; between 4.8 and 8, 7.5, 7, 6.5, 6, 5.5, 5.4, 5.3, 5.2, 5.1, 5, or 4.9; between 4.9 and 8, 7.5, 7, 6.5, 6, 5.5, 5.4, 5.3, 5.2, 5.1, or 5; between 5 and 8, 7.5, 7, 6.5, 6, 5.5, 5.4, 5.3, 5.2, or 5.1; between 5.1 and 8, 7.5, 7, 6.5, 6, 5.5, 5.4, 5.3, or 5.2; between 5.2 and 8, 7.5, 7, 6.5, 6, 5.5, 5.4, or 5.3; between 5.3 and 8, 7.5, 7, 6.5, 6, 5.5, or 5.4; between 5.4 and 8, 7.5, 7, 6.5, 6, or 5.5; between 5.5 and 8, 7.5, 7, 6.5, or 6; between 6 and 8, 7.5, 7, or 6.5; between 6.5 and 8, 7.5, or 7; between 7 and 8, or 7.5; or between 7.5 and 8).

A suitable temperature may be a temperature of between about 20° C. and about 46° C. (e.g., between 20° C. and 46° C., 44° C., 42° C., 40° C., 38° C., 36° C., 34° C., 32° C., 30° C., 28° C., 26° C., 24° C., or 22° C.; between 22° C. and 46° C., 44° C., 42° C., 40° C., 38° C., 36° C., 34° C., 32° C., 30° C., 28° C., 26° C., or 24° C.; between 24° C. and 46° C., 44° C., 42° C., 40° C., 38° C., 36° C., 34° C., 32° C., 30° C., 28° C., or 26° C.; between 26° C. and 46° C., 44° C., 42° C., 40° C., 38° C., 36° C., 34° C., 32° C., 30° C., or 28° C.; between 28° C. and 46° C., 44° C., 42° C., 40° C., 38° C., 36° C., 34° C., 32° C., or 30° C.; between 30° C. and 46° C., 44° C., 42° C., 40° C., 38° C., 36° C., 34° C., or 32° C.; between 32° C. and 46° C., 44° C., 42° C., 40° C., 38° C., 36° C., or 34° C.; between 36° C. and 46° C., 44° C., 42° C., 40° C., or 38° C.; between 38° C. and 46° C., 44° C., 42° C., or 40° C.; between 40° C. and 46° C., 44° C., or 42° C., between 42° C. and 46° C. or 44° C.; or between 44° C. and 46° C.).

A suitable feed rate may be a feed rate of between about 0.01 g and about 0.2 g glucose equivalent per g dry cell weight (DCW) per hour.

A suitable pressure may be a pressure of between 0 psig and about 50 psig (e.g., between 0 psig and 50 psig, 40 psig, 30 psig, 20 psig, or 10 psig; between 10 psig and 50 psig, 40 psig, 30 psig, or 20 psig; between 20 psig and 50 psig, 40 psig, or 30 psig; between 30 psig and 50 psig, or 40 psig; or between 40 psig and 50 psig).

A suitable oxygenation may be an aeration rate of between about 0.1 volumes of oxygen per liquid volume in the fermentor per minute (vvm) and about 2.1 vvm (e.g., between 0.1 vvm and 2.1 vvm, 1.9 vvm, 1.7 vvm, 1.5 vvm, 1.3 vvm, 1.1 vvm, 0.9 vvm, 0.7 vvm, 0.5 vvm, or 0.3 vvm; between 0.3 vvm and 2.1 vvm, 1.9 vvm, 1.7 vvm, 1.5 vvm, 1.3 vvm, 1.1 vvm, 0.9 vvm, 0.7 vvm, or 0.5 vvm; between 0.5 vvm and 2.1 vvm, 1.9 vvm, 1.7 vvm, 1.5 vvm, 1.3 vvm, 1.1 vvm, 0.9 vvm, or 0.7 vvm; between 0.7 vvm and 2.1 vvm, 1.9 vvm, 1.7 vvm, 1.5 vvm, 1.3 vvm, 1.1 vvm, or 0.9 vvm; between 0.9 vvm and 2.1 vvm, 1.9 vvm, 1.7 vvm, 1.5 vvm, 1.3 vvm, or 1.1 vvm; between 1.1 vvm and 2.1 vvm, 1.9 vvm, 1.7 vvm, 1.5 vvm, or 1.3 vvm; between 1.3 vvm and 2.1 vvm, 1.9 vvm, 1.7 vvm, or 1.5 vvm; between 1.5 vvm and 2.1 vvm, 1.9 vvm, or 1.7 vvm; between 1.7 vvm and 2.1 vvm or 1.9 vvm; or between 1.9 vvm and 2.1 vvm).

A suitable fermentation duration may be a fermentation duration of between about 10 hours and about 500 hours (e.g., between 10 hours and 500 hours, 400 hours, 300 hours, 200 hours, 100 hours, 50 hours, 40 hours, 30 hours, or 20 hours; between 20 hours and 500 hours, 400 hours, 300 hours, 200 hours, 100 hours, 50 hours, 40 hours, or 30 hours; between 30 hours and 500 hours, 400 hours, 300 hours, 200 hours, 100 hours, 50 hours, or 40 hours; between 40 hours and 500 hours, 400 hours, 300 hours, 200 hours, 100 hours, or 50 hours; between 50 hours and 500 hours, 400 hours, 300 hours, 200 hours, or 100 hours; between 100 hours and 500 hours, 400 hours, 300 hours, or 200 hours; between 200 hours and 500 hours, 400 hours, or 300 hours; between 300 hours and 500 hours, or 400 hours; or between 400 hours and 500 hours).

A suitable fermentation volume may be between about 1 L and about 10,000,000 L (e.g., between 1 L and 10,000,000 L, 5,000,000 L, 1,000,000 L, 500,000 L, 100,000 L, 50,000 L, 10,000 L, 5,000 L, 1,000 L, 500 L, 100 L, 50 L, or 10 L; between 10 L and 10,000,000 L, 5,000,000 L, 1,000,000 L, 500,000 L, 100,000 L, 50,000 L, 10,000 L, 5,000 L, 1,000 L, 500 L, 100 L, or 50 L; between 50 L and 10,000,000 L, 5,000,000 L, 1,000,000 L, 500,000 L, 100,000 L, 50,000 L, 10,000 L, 5,000 L, 1,000 L, 500 L, or 100 L; between 100 L and 10,000,000 L, 5,000,000 L, 1,000,000 L, 500,000 L, 100,000 L, 50,000 L, 10,000 L, 5,000 L, 1,000 L, or 500 L; between 500 L and 10,000,000 L, 5,000,000 L, 1,000,000 L, 500,000 L, 100,000 L, 50,000 L, 10,000 L, 5,000 L, or 1,000 L; between 1,000 L and 10,000,000 L, 5,000,000 L, 1,000,000 L, 500,000 L, 100,000 L, 50,000 L, 10,000 L, or 5,000 L; between 5,000 L and 10,000,000 L, 5,000,000 L, 1,000,000 L, 500,000 L, 100,000 L, 50,000 L, or 10,000 L; between 10,000 L and 10,000,000 L, 5,000,000 L, 1,000,000 L, 500,000 L, 100,000 L, or 50,000 L; between 50,000 L and 10,000,000 L, 5,000,000 L, 1,000,000 L, 500,000 L, or 100,000 L; between 100,000 L and 10,000,000 L, 5,000,000 L, 1,000,000 L, or 500,000 L; between 500,000 L and 10,000,000 L, 5,000,000 L, or 1,000,000 L; between 1,000,000 L and 10,000,000 L, or 5,000,000 L; or between 5,000,000 L and 10,000,000 L).

A suitable fermentation vessel may be any fermentation vessel known in the art. Non-limiting examples of suitable fermentation vessels include culture plates, shake flasks, fermentors (e.g., stirred tank fermentors, airlift fermentors, bubble column fermentors, fixed bed bioreactors, laboratory fermentors, industrial fermentors, or any combination thereof), used at any suitable scale (e.g., small-scale, large-scale) and in any process (e.g., solid culture, submerged culture, batch, fed-batch, or continuous-flow).

### Purification and Post-Processing

Methods for purifying a recombinant component (e.g., from a fermentation broth) to obtain a preparation comprising the recombinant component are well-known in the art, and may be adapted to purify the recombinant produced by a recombinant host cell according to any of the above.

A recombinant component may be purified on the basis of its molecular weight, for example, by size exclusion/exchange chromatography, ultrafiltration through membranes, gel permeation chromatography (e.g., preparative disc-gel electrophoresis), or density centrifugation.

A recombinant component also may be purified on the basis of its surface charge or hydrophobicity/hydrophilicity, for example, by isoelectric precipitation, anion/cation exchange chromatography, isoelectric focusing (IEF), or reverse phase chromatography.

A recombinant component also may be purified on the basis of its solubility, for example, by ammonium sulfate precipitation, isoelectric precipitation, surfactants, detergents, or solvent extraction.

A recombinant component also may be purified on the basis of its affinity to another molecule, for example, by affinity chromatography, reactive dyes, or hydroxyapatite. Affinity chromatography may include the use of an antibody having a specific binding affinity for the recombinant component, or nickel NTA for a His-tagged recombinant protein, or a lectin to bind to a sugar moiety on a recombinant protein, or any other molecule that specifically binds the recombinant component. The recombinant component may carry an epitope or peptide tag that facilitates purification. An epitope or peptide tag may be removed following isolation of the recombinant component (e.g., via protease cleavage).

In embodiments in which the recombinant component according to any of the above is secreted by the recombinant host cell according to any of the above, the recombinant component may be purified directly from the culture medium. In other embodiments, the recombinant component may be purified from a cell lysate.

The recombinant component may be purified to obtain a preparation comprising the recombinant component at a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% relative to other components comprised in the fermentation broth; or to at least 2-fold, at least 3-fold, at least 4-fold, at least 5-fold, at least 6-fold, at least 7-fold, at least 8-fold, at least 9-fold, or at least 10-fold greater abundancy relative to other components comprised in the fermentation broth; or to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% by mass.

The identity of the recombinant component may be confirmed and/or quantified by high performance liquid chromatography (HPLC), Western blot analysis, Eastern blot analysis, polyacrylamide gel electrophoresis, capillary electrophoresis, formation of an enzyme product, disappearance of an enzyme substrate, and 2-dimensional mass spectroscopy (2D-MS/MS) sequence identification.

The recombinant component may be spray dried or concentrated via evaporation (e.g., to obtain a powder).

### Composition Comprising Recombinant Component

In various aspects, provided herein is a composition that comprises or consists essentially of a recombinant component produced by a recombinant host cell according to any of the above and/or a method according to any of the above, wherein the composition comprises a modulated FFA releasing enzyme activity (e.g., activity of any one FFA releasing enzyme disclosed herein or activities of any combination of two or more FFA releasing enzymes disclosed herein) compared to the FFA releasing enzyme activity in a corresponding composition (i.e., a composition that is identical to the composition that is compared to the “corresponding composition” except that the method by which the “corresponding composition” is produced does not comprise at least one step in which a FFA releasing enzyme activity is modulated as provided herein.

The composition may comprise between about 0.1% and about 100% (e.g., between 0.1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%. 4%. 3%. 2%. 1%. 0.9%. 0.8%. 0.7%. 0.6%. 0.5%, or 0.4%; between 0.4% and 100%, 95%. 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, or 0.8%; between 0.8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, or 2%; between 2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, or 4%; between 4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, or 5%; between 5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, or 6%; between 6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 100%, 95%, 90%, 85%, 80%, 75%, 70%, 65%, 60%, 55%, 50%, 45%, 40%, 35%, 30%, 25%, 20%, 15%, 14%, 13%, 12%, or 11%; between 11% and 100%, 95%, 90%, 85%, 80%, 75%, 70%, 65%, 60%, 55%, 50%, 45%, 40%, 35%, 30%, 25%, 20%, 15%, 14%, 13%, or 12%; between 12% and 100%, 95%, 90%, 85%, 80%, 75%, 70%, 65%, 60%, 55%, 50%, 45%, 40%, 35%, 30%, 25%, 20%, 15%, 14%, or 13%; between 13% and 100%, 95%, 90%, 85%, 80%, 75%, 70%, 65%, 60%, 55%, 50%, 45%, 40%, 35%, 30%, 25%, 20%, 15%, or 14%; between 14% and 100%, 95%, 90%, 85%, 80%, 75%, 70%, 65%, 60%, 55%, 50%, 45%, 40%, 35%, 30%, 25%, 20%, or 15%; between 15% and 100%, 95%, 90%, 85%, 80%, 75%, 70%, 65%, 60%, 55%, 50%, 45%, 40%, 35%, 30%, 25%, or 20%; between 20% and 100%, 95%, 90%, 85%, 80%, 75%, 70%, 65%, 60%, 55%, 50%, 45%, 40%, 35%, 30%, or 25%; between 25% and 100%, 95%, 90%, 85%, 80%, 75%, 70%, 65%, 60%, 55%, 50%, 45%, 40%, 35%, or 30%; between 30% and 100%, 95%, 90%, 85%, 80%, 75%, 70%, 65%, 60%, 55%, 50%, 45%, 40%, or 35%; between 35% and 100%, 95%, 90%, 85%, 80%, 75%, 70%, 65%, 60%, 55%, 50%, 45%, or 40%; between 40% and 100%, 95%, 90%, 85%, 80%, 75%, 70%, 65%, 60%, 55%, 50%, or 45%; between 45% and 100%, 95%, 90%, 85%, 80%, 75%, 70%, 65%, 60%, 55%, or 50%; between 50% and 100%, 95%, 90%, 85%, 80%, 75%, 70%, 65%, 60%, or 55%; between 55% and 100%, 95%, 90%, 85%, 80%, 75%, 70%, 65%, or 60%; between 60% and 100%, 95%, 90%, 85%, 80%, 75%, 70%, or 65%; between 65% and 100%, 95%, 90%, 85%, 80%, 75%, or 70%; between 70% and 100%, 95%, 90%, 85%, 80%, or 75%; between 75% and 100%, 95%, 90%, 85%, or 80%; between 80% and 100%, 95%, 90%, or 85%; or between 85% and 100%, 95%, 90%; between 90% and 100% or 95%, orbetween 95% and 100%) by dry mass of the recombinant component.

At standard ambient temperature and conditions (i.e., 20-30° C. and 0.95-1.05 atm), the composition according to any of the above may be a fluid, semi-solid (e.g., gelatinous), solid, or powder. The powder may comprise a moisture content of less than 20%, less than 15%, less than 10%, less than 7%, less than 5%, less than 3%, or less than 1%; or between about 0.1% and about 20% (e.g., between 0.1% and 20%, 15%, 10%, 5%, or 1%; between 1% and 20%, 15%, 10%, or 5%; between 5% and 20%, 15%, or 10%; between 10% and 20%, or 15%; or between 15% and 20%). The powder may be used in powder form, or the powder may be reconstituted with a hydrating agent prior to use, or the powder may be mixed with other dry components (e.g., flour, sugar, minerals, pH or ionic strength adjusting agents) before a hydrating agent is added to the mixture.

The composition according to any of the above may be a variety of products, including, for example, an encapsulate (e.g., a product that encapsulates a therapeutic or nutraceutical for delivery [e.g., a micro- or nano-particle (e.g., a bead, a micelle, a capsule, a hydrogel), a composition with industrial utility (e.g., a dielectric), an adhesive (i.e., a material that forms an adhesive bond; e.g., glue, wallpaper adhesive, wood adhesive, paper adhesive, cork adhesive, chipboard adhesive, surgical/medical glue, cement, mucilage, paste), a coating or facing (e.g., glossy coating, protective coating, varnish, coating for medical tablet, paper coating, painting, leather finishing, textile coating), a spray, a paint or ink or pigment binder for ink, a hard plastic (e.g., bottle, button, window, pen), a medium hard plastic (e.g., bottle, fiber [e.g., yarn]), a soft plastic (e.g., bag, wrap, edible film, waterproof film, contact lens, packaging material), a fabric (e.g., textile, carpet, curtain, clothing), an industrial polymer (i.e., compound used in the manufacture of synthetic industrial materials), a medical diagnostic (see, for example, J. Berger et al. 2004. Europ J of Pharm and Biopharm 57:19, respectively), a gel (e.g., hydrogel for controlled release of a therapeutic, hydrogel for immobilizing a protein (e.g., enzyme)), an implant (e.g., bone-replacing composite, material supporting nerve repair, scaffold for growing cells, prosthetic implant), an article of clothing (e.g., shoe), a lubricant, a piece of furniture, a paper (e.g., paper sheet, paper label, packaging paper, photographic support), a household item (e.g., pot, bowl, plate, cup), and a biological scaffold (i.e., a structure that mimics a biological matrix, sutures, bone-replacing material, material supporting nerve repair, scaffold for growing cells, prosthetic implant, membrane for promoting wound healing, wound dressing, tissue-engineering scaffolding).

### Food Product

The composition according to any of the above may be a food product.

The term “food product” as used herein refers to a composition that can be ingested by a human or an animal for dietary purposes (i.e., without ill health effects but with significant nutritional and/or caloric intake due to uptake of digested material in the gastrointestinal tract), including a domesticated animal (e.g., dog, cat), farm animal (e.g., cow, pig, horse), and wild animal (e.g., non-domesticated predatory animal). The term includes compositions that may be combined with or added to one or more other ingredients to make a food product that can be ingested by a human or an animal.

The food product may be a supplemented food product (i.e., a conventional food product that is supplemented with the recombinant component according to any of the above), or may be a substitute food product (i.e., a food product that resembles a conventional food product and that can be used in place of the conventional food product), selected from any of the food product categories defined by the National Health and Nutrition Examination Survey (NHANES).

Non-limiting examples of NHANES food product categories include snack foods and gums (e.g., snack bars, crackers, salty snacks from grain products, chewing gums); breads, grains, and pastas (e.g., oat breads and rolls, cornbread, corn muffins, tortillas, flour and dry mixes, biscuits, multi-grain breads and rolls, whole wheat breads and rolls, pastas, rye breads and rolls, cracked wheat breads and rolls, white breads and rolls); beverages (e.g., beers and ales, beverage concentrates, beverages, energy drinks, sports drinks, fluid replacements, soft drinks, carbonated beverages, juices, wines, beers, cocktails, nutrition drinks, nutrition powders, protein-enriched beverages, coffee, tea); sweets and desserts (e.g., cakes, candies, chips, cookies, cobblers, pastries, ices or popsicles, muffins, pies, sugar replacements or substitutes, syrups, honey, jellies, jams, preserves, salads, crepes, Danish, breakfast pastries, doughnuts); breakfast foods (e.g., cereal grains, cereal, rice, French toast, pancakes, waffles, coffee cake); salad dressings, oils, sauces, condiments (e.g., cooking fats, vegetable oils, salad dressings, tomato sauces, gravies); potatoes (e.g., potato salad, potato soups, chips and sticks, fried potatoes, mashed potatoes, stuffed potatoes, puffs); and soups (e.g., vegetable soups, vegetable broths), meals, main dishes, proteins (e.g., meat substitutes), and seafoods.

The food product according to any of the above may be a supplemented dairy product (i.e., a conventional dairy product that is supplemented with the recombinant component according to any of the above) or a substitute dairy product (i.e., a food product that resembles a conventional dairy product). The term “dairy product” as used herein refers to milk (e.g., whole milk [at least 3.25% milk fat], partly skimmed milk [from 1% to 2% milk fat], skim milk [less than 0.2% milk fat], cooking milk, condensed milk, flavored milk, goat milk, sheep milk, dried milk, evaporated milk, milk foam), and products derived from milk, including but not limited to yogurt (e.g., whole milk yogurt [at least 6 grams of fat per 170 g], low-fat yogurt [between 2 and 5 grams of fat per 170 g], nonfat yogurt [0.5 grams or less of fat per 170 g], greek yogurt [strained yogurt with whey removed], whipped yogurt, goat milk yogurt, Labneh [labne], sheep milk yogurt, yogurt drinks [e.g., whole milk Kefir, low-fat milk Kefir], Lassi), cheese (e.g., whey cheese such as ricotta; pasta filata cheese such as mozzarella; semi-soft cheese such as Havarti and Muenster; medium-hard cheese such as Swiss and Jarlsberg and halloumi; hard cheese such as Cheddar and Parmesan; washed curd cheese such as Colby and Monterey Jack; soft ripened cheese such as Brie and Camembert; fresh cheese such as cottage cheese, feta cheese, cream cheese, paneer, and curd), processed cheese, processed cheese food, processed cheese product, processed cheese spread, enzyme-modulated cheese; cold-pack cheese), dairy-based sauces (e.g., salad dressing, bechamel sauce, fresh sauces, frozen sauces, refrigerated sauces, shelf stable sauces), dairy spreads (e.g., low-fat spread, low-fat butter), cream (e.g., dry cream, heavy cream, light cream, whipping cream, half-and-half, coffee whitener, coffee creamer, sour cream, creme fraiche), frozen confections (e.g., ice cream, smoothie, milk shake, frozen yogurt, sundae, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, milk protein concentrate (e.g., milk protein concentrate, whey protein concentrate, demineralized whey protein concentrate, β-lactoglobulin concentrate, α-lactalbumin concentrate, glycomacropeptide concentrate, casein concentrate), milk protein isolate (e.g., milk protein isolate, whey protein isolate, demineralized whey protein isolate, β-lactoglobulin protein isolate, α-lactalbumin protein isolate, glycomacropeptide protein isolate, casein protein isolate), nutritional supplements, texturizing blends, flavoring blends, coloring blends, ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, bars (e.g., nutrition bars, protein bars), and fermented dairy products (e.g., yoghurt, cheese, sour cream, cultured buttermilk, cultured butter, cultured butter oil).

The food product according to any of the above may be a supplemented animal meat or animal meat product (i.e., a conventional animal meat or animal meat product that is supplemented with the recombinant component according to any of the above produced by the recombinant host cell according to any of the above and/or a method according to any of the above) or a substitute animal meat or animal meat product (i.e., a food product that resembles a conventional animal meat or animal meat product). Non-limiting examples of animal meats and animal meat products include flesh obtained from skeletal muscle or from other organs (e.g., kidney, heart, liver, gallbladder, intestine, stomach, bone marrow, brain, thymus, lung, tongue), or parts thereof, obtained from an animal. The animal meat may be dark or white meat. Non-limiting examples of animals from which animal meat or animal meat product can be obtained include cattle, lamb, mutton, horse, poultry (e.g., chicken, duck, goose, turkey), fowl (e.g., pigeon, dove, grouse, partridge, ostrich, emu, pheasant, quail), fresh or salt water fish (e.g., catfish, tuna, spearfish, shark, halibut, sturgeon, salmon, bass, muskie, pike, bowfin, gar, eel, paddlefish, bream, carp, trout, walleye, snakehead, crappie, sister, mussel, scallop, abalone, squid, octopus, sea urchin, cuttlefish, tunicate), crustacean (e.g., crab, lobster, shrimp, barnacle), game animal (e.g., deer, fox, wild pig, elk, moose, reindeer, caribou, antelope, zebra, squirrel, marmot, rabbit, bear, beaver, muskrat, opossum, raccoon, armadillo, porcupine, bison, buffalo, boar, lynx, bobcat, bat), reptile (e.g., snakes, turtles, lizards, alligators, crocodiles), any insect or other arthropod, rodent (nutria, guinea pig, rat, mice, vole, groundhog, capybara), kangaroo, whale, and seal. The animal meat or animal meat product may be ground, chopped, shredded, or otherwise processed, and uncooked, cooking, or cooked.

The food product according to any of the above may be a supplemented egg product (i.e., a conventional egg or egg product that is supplemented with the recombinant component according to any of the above) or a substitute egg or egg product (i.e., a food product that resembles a conventional egg or egg product). Non-limiting examples of eggs or egg products include whole egg (e.g., liquid whole egg, spray-dried whole egg, frozen whole egg), egg white (e.g., liquid egg white, spray-dried egg white, frozen egg white), egg yolk, egg dishes, egg soups, mixtures made with egg whites, mixtures made with egg substitutes, mayonnaise, custard, and salad dressings.

Resemblance of a substitute food product provided herein to a conventional food product may be due to any physical attribute, chemical/biological attribute, sensory attribute, and functional attribute, and any combination thereof.

The food product according to any of the above may be a pet food or animal feed.

The food product according to any of the above may be essentially free of any protein other than a recombinant protein or recombinant proteins comprised in the composition according to any of the above.

The food product according to any of the above may be essentially free of any recombinant protein other than a recombinant protein or recombinant proteins contained in the composition according to any of the above.

The food product according to any of the above may be essentially free of any recombinant milk protein other than a recombinant protein or recombinant proteins contained in the composition according to any of the above.

The food product according to any of the above may be essentially free of a component found in a mammal-produced milk (e.g., cow milk, goat milk, sheep milk, human milk, buffalo milk, yak milk, camel milk, llama milk, alpaca milk, horse milk, donkey milk), or may comprise a lower concentration of at least one component found in a mammal-produced milk. Non-limiting examples of components found in mammal-derived milk include lactose, saturated fat, cholesterol, native milk proteins, and native milk lipids. The food product may be essentially free of any milk protein other than a recombinant milk protein or recombinant milk proteins contained in the composition according to any of the above.

The food product according to any of the above may be essentially free of a component obtained from an animal (i.e., a component that is native to an animal, including animal products [i.e., parts of an animal that are consumables or typically prepared for consumption by humans; e.g., animal meat, animal fat, animal blood], animal byproducts [i.e., products that are typically not consumable by themselves but are the byproducts of slaughtering animals for consumption; e.g., animal bones, animal carcasses, and constituents isolated therefrom], products produced by an animal [e.g., mammal-derived milk, chicken eggs, bee honey], and consumables produced therefrom [e.g., gelatin, rennet, whey proteins extracted from mammal-derived milk, casein extracted from mammal-derived milk, milk lipid extracted from mammal-derived milk, animal lipids, animal proteins]), or comprise 2% or less by mass of such component.

A variety of recipes exist for preparing a food product, and any such recipe may be used to produce a food product according to any of the above. The recombinant component may be used in such recipe in purified/isolated form or comprised in a fermentation broth or preparation obtained by a method according to any of the above.

### Cosmetic or Personal Care Product

The composition according to any of the above may be a cosmetic or personal care product.

The term “cosmetic or personal care product” as used herein refers to a composition that upon application to a body surface (i.e., an exposed area of a human body, such as skin, hair, nail, tooth, and tissues of the oral cavity [e.g., gums]) confers a perceived or actual beautifying or hygienizing effect. Non-limiting examples of cosmetic or personal care products include anti-wrinkling treatments (i.e., compositions used for tensioning [e.g., smoothing out of skin, reducing wrinkles in skin, removing fine lines in skin]), anti-aging treatments (i.e., compositions used for removing signs of aging [e.g., wrinkles, fine lines, manifestations of photodamage (e.g., sun spots)]), sun protection (i.e., compositions used to protect against UV exposure), anti-burn treatments (i.e., compositions used for soothing burns [e.g., sunburns]), anti-acne treatments (i.e., compositions that are effective in the treatment of acne and/or the symptoms associated therewith), skin cleansers (i.e., compositions used for cleaning skin and/or skin pores [e.g., nose strips for pore cleaning]), anti-dandruff treatments (i.e., compositions used for reducing or eliminating dandruff), anti-body odor treatments (i.e., compositions used for reducing or eliminating body odor), self-tanning treatments (i.e., compositions used for darkening skin color), skin whitening treatments (i.e., compositions used for bleaching/depigmenting skin color), hair coloring treatments (i.e., compositions used for coloring hair), lotions (e.g., skin lotions, body care lotions, wash lotions, moisture retention lotions, pre-shave lotions, after-shave lotions), pastes (e.g., washing pastes), ointments, balms, salves, masks, creams (e.g., water in oil creams, oil in water creams, day creams, night creams, eye creams, skin creams, face creams, anti-wrinkle creams, sun protection creams, moisture retention creams, after-shave creams, skin bleaching creams, self-tanning creams, vitamin creams, moisturizing creams, massage creams), milks (e.g., body milks, cleansing milks), gels (e.g., anhydrous gels, shower gels), Eau de Toilette, soaps (e.g., transparent soaps, luxurious soaps, deodorant soaps, cream soaps, baby soaps, skin protection soaps, abrasive soaps, syndets, pasty soaps, soft soaps, peeling soaps), skin peeling treatments, liquid washes, shower and bath preparations (e.g., wash lotions, shower baths, shower gels, foam baths, oil baths, scrub preparations), foams (e.g., shaving foams, foam baths), deodorants, hair care products (e.g., shampoos, conditioners, hair mousses, hair colorants, hair sprays, rinse-off lotions, hair gels, hair emulsions, hair laquers, hair tonics), lip glosses, sprays (e.g., hair sprays, pump sprays, sprays containing blowing agents), treatments for skin defects (e.g., dermatitis, weals, chaps, blemishes, cracks, scars, freckles, moles, rashes, blisters, pustules), toners, cleaning tissues, sanitary towels, tampons, nappies, repellents, make-up products (e.g., studio pigments, mascara, eye shadows, eyeliners, eye liner pens, rouges, face powders, eyebrow pencils, lipsticks, foundations, tinted creams, concealer sticks, blemish sticks, blushes), sticks (e.g., lipsticks, concealer sticks, blemish sticks), hair removing agents, hand cleaning products, intimate hygiene products, foot care products, baby care products, and oral hygiene products (e.g., chewing gums, mouthwashes, toothpastes, gum-cleaning agents, denture adhesives, denture fixatives).

The cosmetic or personal care product according to any of the above may be essentially free of any protein other than the recombinant protein or the recombinant proteins contained in the composition according to any of the above.

The cosmetic or personal care product according to any of the above may be essentially free of any recombinant protein other than the recombinant protein or the recombinant proteins contained in the composition according to any of the above.

The cosmetic or personal care product according to any of the above may be essentially free of any recombinant milk protein other than the recombinant protein or the recombinant proteins contained in the composition according to any of the above.

The cosmetic or personal care product according to any of the above may be essentially free of a component found in a mammal-produced milk (e.g., cow milk, goat milk, sheep milk, human milk, buffalo milk, yak milk, camel milk, llama milk, alpaca milk, horse milk, donkey milk), or may comprise a lower concentration of at least one component found in a mammal-produced milk. Non-limiting examples of components found in mammal-derived milk include lactose, saturated fat, cholesterol, native milk proteins, and native milk lipids. The cosmetic or personal care product may be essentially free of any milk protein other than the milk protein comprised in the recombinant protein or the recombinant proteins contained in the composition according to any of the above.

The cosmetic or personal care product according to any of the above may be essentially free of a component obtained from an animal (i.e., a component that is native to an animal, including animal products [i.e., parts of an animal that are consumables or typically prepared for consumption by humans; e.g., animal meat, animal fat, animal blood], animal byproducts [i.e., products that are typically not consumable by themselves but are the byproducts of slaughtering animals for consumption; e.g., animal bones, animal carcasses, and constituents isolated therefrom], products produced by an animal [e.g., mammal-derived milk, chicken eggs, bee honey], and consumables produced therefrom [e.g., gelatin, rennet, whey proteins extracted from mammal-derived milk, casein extracted from mammal-derived milk, milk lipid extracted from mammal-derived milk, animal lipids, animal proteins]), or comprise 2% or less by mass of such component.

The cosmetic or personal care product according to any of the above may be essentially free of a component derived from petroleum.

### Recombinant Host Cell Producing FFA Releasing Enzyme Activity

In various aspects, provided herein is a recombinant host cell that comprises a recombinant expression construct encoding a FFA releasing enzyme, and that comprises an increased production and/or activity of the FFA releasing enzyme compared to a corresponding host cell (i.e., a host cell that is essentially identical to the recombinant host cell except that it does not comprise the recombinant expression construct encoding the FFA releasing enzyme).

The recombinant host cell may be derived from any wild type unicellular organism, including any bacterium, yeast, filamentous fungus, archaea, unicellular protista, unicellular animals, unicellular plants, unicellular algae, protozoa, and chromista, or from a genetic variant (e.g., mutant) thereof, as well as from any generally recognized as safe (GRAS) industrial host cell, and including any organism disclosed herein (e.g., Trichoderma reesei, Aspergillus niger, Trichoderma citrinoviride, Myceliophthora thermophila).

The recombinant host cell according to any of the above may comprise an increased production and/or activity of a FFA releasing enzyme activity selected from the group consisting of FFA releasing enzymes comprising UniProt sequence #G0RH85, G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, G0RVD2, and G0R8A6, and homologs thereof, and combinations thereof; wherein the increased production and/or activity of the FFA releasing enzyme is an increase in production and/or activity of at least 5%, at least 10%, at least 15%, at least 20%, at least 25%, at least 50%, at least 75%, at least 100%, at least 150%, at least 200%, at least 300%, at least 400%, at least 500%, at least 600%, at least 700%, at least 800%, at least 900%, or at least 1,000%.

In various aspects, provided herein is a method for obtaining a recombinant host cell that comprises a recombinant expression construct encoding a FFA releasing enzyme, wherein the method comprises: i) obtaining a polynucleotide according to any of the above, or a recombinant expression construct according to any of the above, or a recombinant vector according to any of the above, wherein the recombinant protein coding sequence of the polynucleotide, recombinant expression construct, or recombinant vector encodes a FFA releasing enzyme activity selected from the group consisting of FFA releasing enzymes comprising UniProt sequence #G0RH85, G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, G0RVD2, and G0R8A6, and homologs thereof; and ii) introducing the polynucleotide, expression construct, or recombinant vector into a host cell (e.g., any of the host cells disclosed herein; using any of the methods disclosed herein) to obtain a recombinant host cell comprising an increased production and/or activity of the FFA releasing enzyme.

In various aspects, provided herein is a method for producing a FFA releasing enzyme, wherein the method comprises: obtaining a recombinant host cell that comprises a recombinant expression construct encoding the FFA releasing enzyme, and that comprises an increased production and/or activity of the FFA releasing enzyme compared to a corresponding host cell; culturing the recombinant host cell in a culture medium under conditions suitable for production and/or secretion of the FFA releasing enzyme; and optionally purifying the FFA releasing enzyme.

### EXAMPLES

The following examples are included to illustrate specific embodiments of this disclosure. The techniques disclosed in the examples represent techniques discovered by the inventors to function well in the methods and processes of this disclosure; however, those of skill in the art should, in light of the present disclosure, appreciate that many changes can be made in the specific embodiments that are disclosed and still obtain a like or similar result without departing from the spirit and scope of the disclosure. Therefore, all matter set forth or shown in the examples is to be interpreted as illustrative and not in a limiting sense.

### Example 1: Expression Analysis of Recombinant Protein Preparations

The presence of G0RMI3, G0RGQ0 and G0RLH4 mRNA transcripts in various recombinant Trichoderma reesei host cells (i.e., various Trichoderma reesei host cells comprising a recombinant expression construct encoding Bos taurus β-lactoglobulin) was determined by fermenting the recombinant Trichoderma reesei host cells in 2 L tanks under a variety of conditions suitable for production and secretion of the recombinant β-lactoglobulin. Biomass samples were taken at various time points during fermentations, and flash frozen in liquid nitrogen. RNA was extracted from the samples, the RNA quality was checked with agarose gel electrophoresis, and the RNA was submitted for RNA sequencing and read processing/analysis. Expression levels were assessed for transcripts encoding G0RMI3, G0RGQ0, and G0RLH4 proteins.

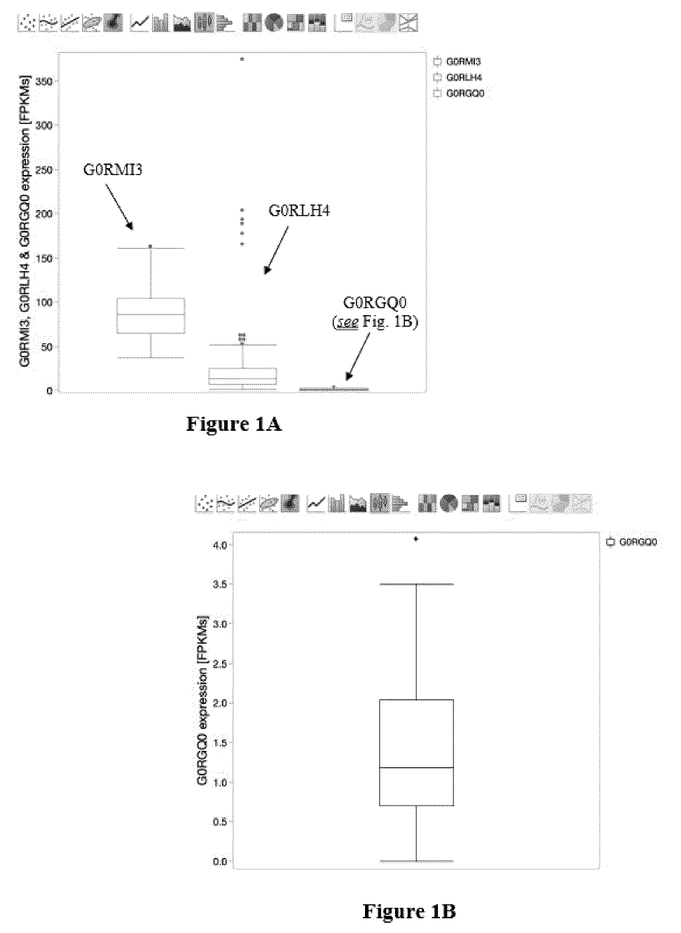

As shown in FIGS. 1A and 1B, fermentations of recombinant Trichoderma reesei host cells producing recombinant protein showed detectable expression of G0RMI3, G0RGQ0, and G0RLH4 transcripts.

### Example 2: Removal of FFA Releasing Activity Via Inhibition

A recombinant Trichoderma reesei host cell capable of producing a recombinant β-lactoglobulin and comprising an essentially eliminated cutinase (e.g., cut1 (UniProt #G0RH85)) activity (“cutinase knockout recombinant host cell”) was generated and fermented as described in PCT patent publication WO2020/081789. Recombinant β-lactoglobulin was isolated from clarified fermentation broths of the recombinant Trichoderma reesei host cell based on charge (i.e., electrostatic interaction), and spray dried to obtain a β-lactoglobulin powder preparation.

To remove from the β-lactoglobulin powder preparation activity of FFA releasing enzymes that comprise a serine residue, such as G0RMI3, G0RGQ0, and G0RLH4 proteins, the powder preparation was dissolved in water to a final concentration of 40 g/L proteins. Two 1-mL samples of the solution were taken as a Sample 1 and a Sample 2. To Sample 1 was added 20 μL of dimethyl sulfoxide (DMSO) to a final concentration of 2%. To Sample 2 was added 20 μL of 0.1 mM phosphonate substrate inhibitor Thermo ActivX TAMRA-FP luorophosphonate (ThermoFisher Scientific catalog number 88318; a member of the phosphonate inhibitors of hydrolases comprising a nucleophilic serine in their active site (Simon & Cravatt. 2010. J. Biol. Chem. 285(15):11051-11055)) in DMSO to a final concentration of 2 μM Thermo ActivX TAMRA-FP fluorophosphonate and 2% DMSO. The samples were incubated for 18.5 hours at 21° C.

Para-phenyl (pNP) acyl ester hydrolyzing enzyme activities comprised in Sample 1 and Sample 2 were determined by first diluting each sample with equal volume of 0.4 M 4-(2-hydroxyethyl)-1-piperazineethanesulfonic acid (HEPES), pH 7, then diluting serially up to 1/128 fold with 0.2 M HEPES pH7, mixing with equal volumes of 0.3 mM pNP acyl ester (pNP-butyrate, pNP-octanoate, pNP-laurate, or pNP-palmitate) in 10% DMSO, and incubating at 30° C. The reaction mixtures were measured hourly (from 1 to 6 hours) for absorbance at 348 nm by a SpectraMax M3 plate reader from Molecular Devices (San Jose, California) in UV-star plates from Greiner Bio-one (Monroe, North Carolina; catalogue number G55801).

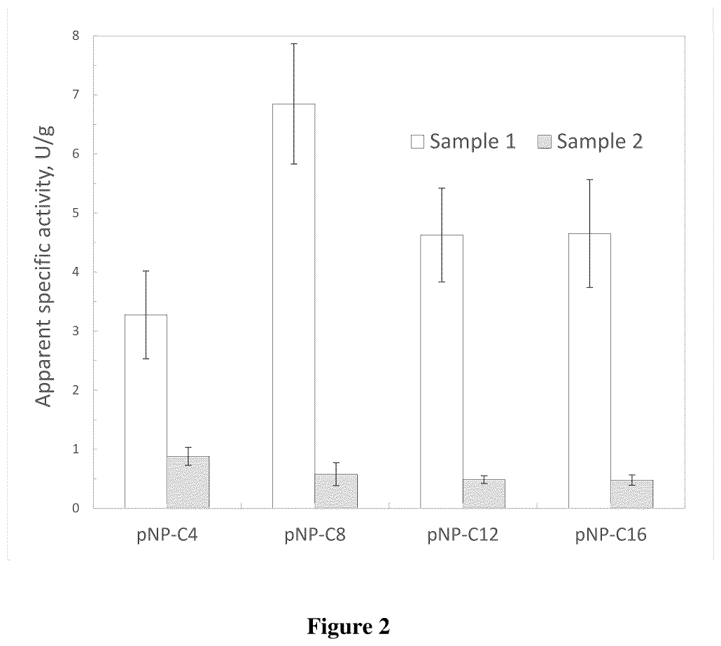

As shown in FIG. 2, Sample 1 produced para-nitrophenol from the hydrolysis of the four pNP acyl esters, which co-produced corresponding FFAs and is indicative of the presence of a lipase (as an esterase) activity in Sample 1. Such generation of FFAs (i.e., fatty acid ester hydrolyzing enzyme activity) was essentially eliminated in Sample 2.

### Example 3: Removal of Esterase Activity Via Purification

A recombinant Trichoderma reesei host cell capable of producing a recombinant β-lactoglobulin and comprising an essentially eliminated cutinase (e.g., cut1 (UniProt #G0RH85)) activity (“cutinase knockout recombinant host cell”) was generated and fermented as described in PCT patent publication WO2020/081789. Recombinant β-lactoglobulin was isolated from clarified fermentation broths of the recombinant Trichoderma reesei host cell based on charge (i.e., electrostatic interaction), and spray dried to obtain a recombinant β-lactoglobulin powder preparation.

The recombinant β-lactoglobulin powder preparation was re-dissolved in water to 40 or 200 g/L concentration, and 2.5 or 7.5 mL, respectively, of the solution was reacted with 80 or 20 μL of 0.1 mM ActivX™ desthiobiotin-fluorophosphonate serine hydrolase probe (DTB-FP, catalog number 88317, ThermoFisher Scientific, Waltham, MA) in DMSO at 21° C. DTB-FP covalently binds FFA releasing enzymes that comprise a nucleophilic serine in their active sites by bonding to the phosphorus and the side chain oxygen of the serine. After 8 hours, 100 μL of hydrated Pierce™ high capacity streptavidin agarose (SA-A; ThermoFisher Scientific, Waltham, MA) was added, and the reaction suspension was incubated at 21° C. (FFA releasing enzymes with attached DTB-FP bind to SA-A via biotin-avidin binding). After 21 hours, the suspension was centrifuged for 2 minutes at 1,000 relative centrifugal force (rcf), and the supernatant (comprising the recombinant β-lactoglobulin) was separated from the pellet (comprising bound FFA releasing enzymes that comprise a serine residue in their catalytic domain, such as G0RMI3, G0RGQ0, and G0RLH4 proteins).

### Example 4: Production of Recombinant G0RMI3, G0RGQ0 and G0RLH4 Proteins

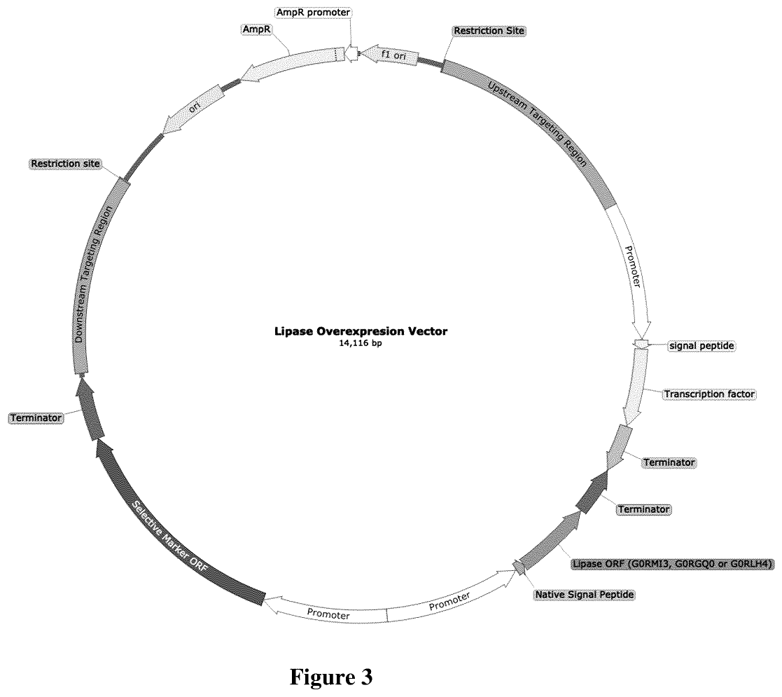

For recombinant G0RLH4 and G0RGQ0 protein production in Trichoderma reesei, recombinant vectors were constructed using genetic engineering methods known in the art. The general structure of the recombinant vectors is shown in FIG. 3. The recombinant vectors comprised a recombinant expression construct comprising a protein coding sequence encoding G0RLH4 or G0RGQ0 protein, operably linked to a N-terminal G0RLH4 or G0RGQ0 native secretion signal sequence, respectively, and under control of a pSES promoter and a pdc1 terminator. The recombinant expression construct further comprised a polynucleotide encoding a synthetic transcription factor to drive expression of the G0RLH4 or G0RGQ0 expression constructs. The recombinant vector further comprised a polynucleotide that could direct integration of the recombinant expression construct at the eg11 locus in the genome of a Trichoderma reesei host cell, selection markers for selection of bacterial and/or fungal transformants, and a bacterial origin of replication. The bacterial selection markers and origin of replication were removed from the recombinant vector via restriction enzyme digestion prior to transformation of the recombinant vector into a Trichoderma reesei host cell.

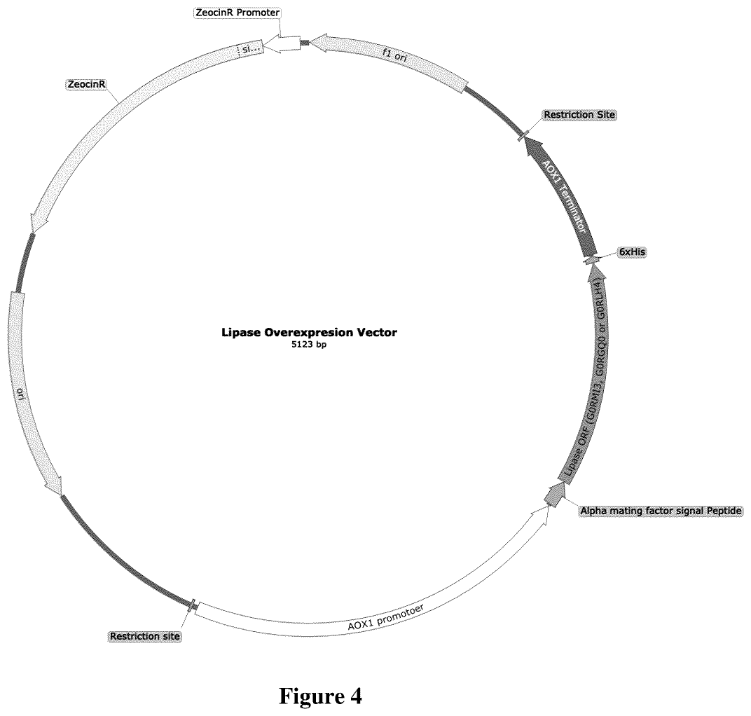

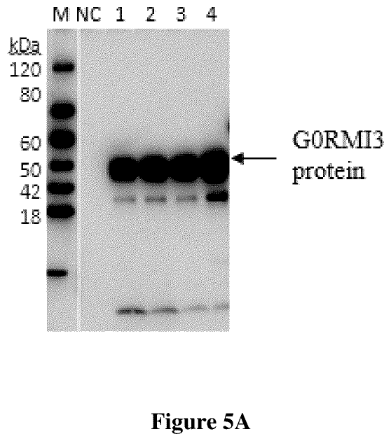

For recombinant G0RMI3 protein production in Pichia pastoris, a recombinant vector was constructed using genetic engineering methods known in the art. The general structure of the recombinant vector is shown in FIG. 4. The recombinant vector comprised a recombinant expression construct comprising a protein coding sequence encoding G0RMI3 protein, operably linked to a N-terminal pre-pro secretion signal sequence of the alpha mating factor of Saccharomyces cerevisiae and to a C-terminal 6×-His tag, under control of an AOX1 methanol-inducible promoter and terminator. The recombinant vector further comprised a selection marker for selection of bacterial and/or fungal transformants, and a bacterial origin of replication. The bacterial selection markers and origin of replication were removed from the recombinant vector via restriction enzyme digestion prior to transformation of the recombinant vector into a Pichia pastoris host cell.

The recombinant vectors were transformed into Trichoderma reesei or Pichia pastoris host cells (e.g., by using a heat-shock protocol), and transformants were selected by growth on minimal media or antibiotics for positive selection. The transformants were grown in expression media in 24-well plates, and supernatants were harvested for further analysis. Recombinant host cells that comprised an integrated copy of a recombinant expression construct according to any of the above, and that secreted recombinant G0RMI3, G0RLH4, or G0RGQ protein, were identified by SDS-PAGE or Western blot gel analyses of fermentation broth samples.

The recombinant G0RMI3 protein was purified from fermentation broth using an affinity column (e.g., a HisTrap HP column (GE Healthcare, Piscataway, NJ)), and then eluted at 0.16 mg/mL concentration (as determined by Bradford assay) in buffered solution of 50 mM Tris-HCL, 150 mM NaCL, 10% glycerol at pH8.

The recombinant G0RGQ0 and G0RLH4 proteins were not purified, but rather supernatants from 5 day old shake flasks were used in subsequent experiments. Shake flasks were inoculated with 1×10⁸ spores into 100 ml of shake flask minimal media.

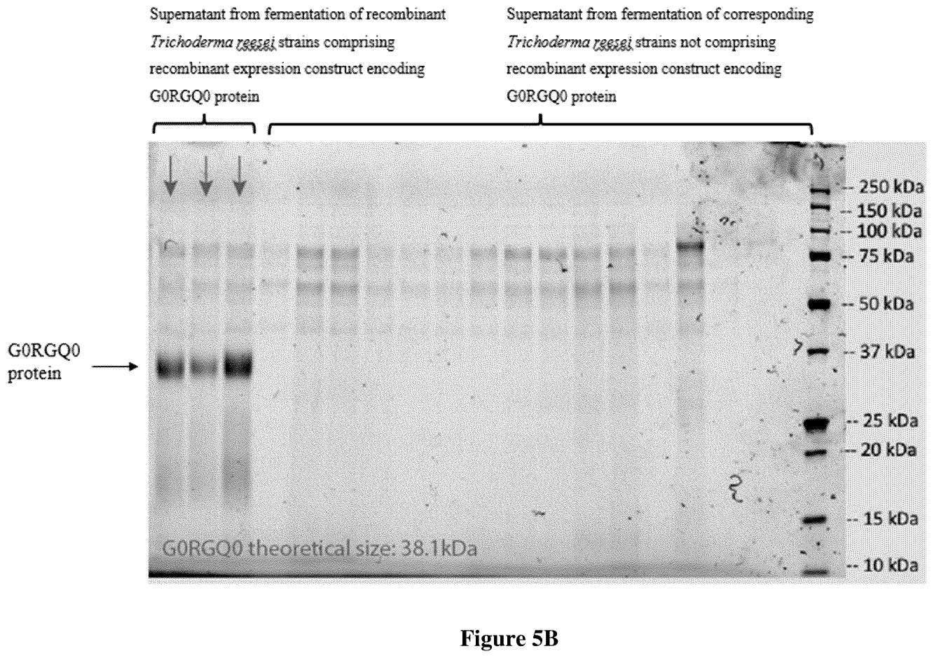

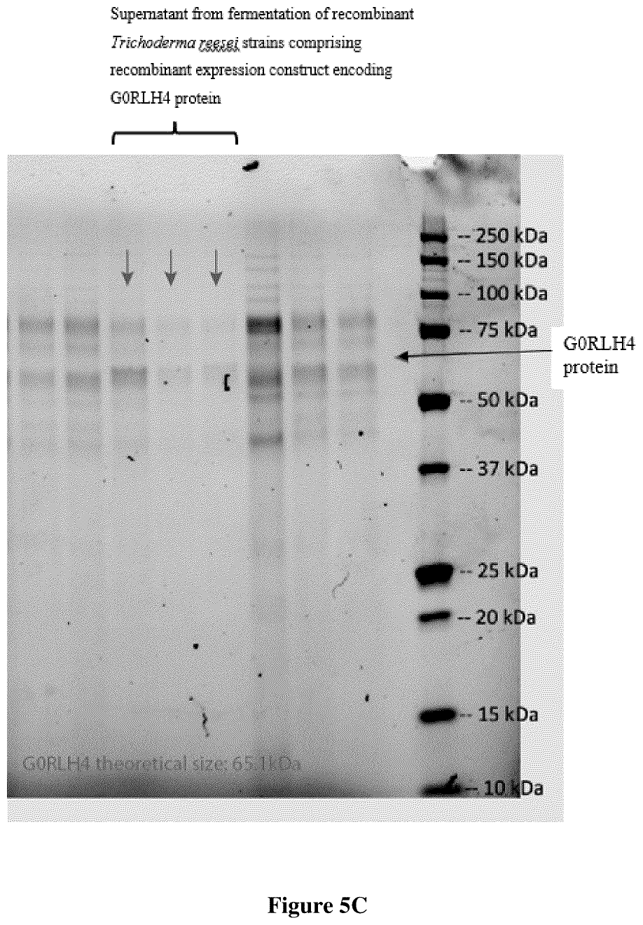

As shown in FIGS. 5A-5C, the recombinant strains produced G0RMI3, G0RGQ0, and G0RLH4 proteins.

### Example 5: Analysis of FFA Releasing Activity of G0RMI3, G0RGQ0, and G0RLH4 Proteins Using Lipolytic Assay

FFA releasing activities of G0RMI3, G0RGQ0, and G0RLH4 proteins were demonstrated using a method based on detection of fluorescent signal caused by FFA binding and concentration of rhodamine B (Kouger & Jaeger. 1987. Appl Env Microbiol. 53(1):211-213). To this end, wells of a 24-well culture plate were each filled with 3 mL of an agar gel comprising 1% weight by volume agar, 10 mL/L of sunflower coconut oil blend, and 5 mg/L rhodamine B. Onto the agar was then pipetted a 10 uL sample of the G0RMI3 protein preparation of Example 3, a 20 uL sample of the G0RGQ0 or G0RLH4 culture supernatant of Example 3, or an equivalent volume of a negative control (i.e., supernatant of a Trichoderma reesei strain that did not comprise any of the expression constructs of Example 3). The 24-well plate was incubated at 30° C. for 48 hours, and then illuminated with a UV transilluminator to determine whether FFAs were present.

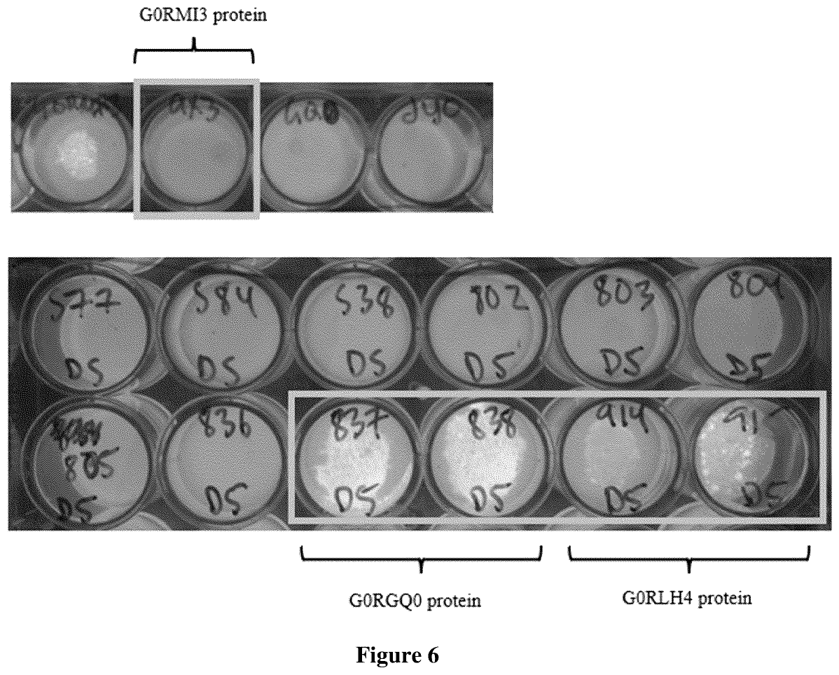

As shown in FIG. 6, fluorescent signal was observed for wells containing G0RMI3, G0RGQ0, or G0RLH4 protein, but not from negative control wells, indicating that G0RMI3, G0RGQ0, and G0RLH4 proteins can cause enzymatic release of FFAs from sunflower/coconut oil substrate.

### Example 6: Analysis of FFA Releasing Activity of G0RMI3 Protein Using Ice Cream Rancidity Assay

FFA releasing activities of G0RMI3 protein was demonstrated by evaluating ice creams produced from a preparation comprising the protein for rancidity. To this end, an ice cream mix was produced that comprised sugar, maltodextrin, salt, minerals, gum, and Bos taurus whey protein isolate dissolved in water. To this mix, 16% by mass of lipids (i.e., emulsified mono- and diglycerides obtained from soy, melted coconut oil, and melted sunflower oil) were added, and the mixture was blended at 20,000 rpm for 30 seconds in an Ultraturrax (IKA Works, Wilmington, NC). The blend was transferred to a Hot Mix Pro container equipped with scrape surface paddle, and incubated with a hold temp of 82° C. and hold time of 25 seconds, on 90 rpm. The finished ice cream base was pasteurized and homogenized (2-stage: 180/30 bar=210 bar), before approximately 207 g of it was added to a 250 mL Schott bottle, and incubated in a water bath at 55° C. To the blend was added 15 ug of G0RMI3 protein, and the mixture was mixed using an Ultraturrax at 16,000 rpm for at least 60 seconds, and then incubated in a water bath at 55° C. After 4 hours, the samples were cooled and stored at 4° C. for 7-8 days. The ice cream was evaluated by sensory experts at time points 0 hour, 4 hours, 24 hours, and 6-8 days. Samples that comprised the G0RMI3 protein were found to have rancid smell and/or taste.

### Example 7: Analysis of FFA Releasing Activity of G0RMI3 Protein Using Yogurt Rancidity Assay

FFA releasing activities of G0RMI3 protein was demonstrated by evaluating yogurts produced from preparations comprising the proteins for rancidity. To this end, a first mixture of 50 mg of YCX-11 yogurt culture (Chr. Hansen Inc., Hsrsholm, Denmark) and 80 g of whole milk was mixed on a stir plate at low speed for at least 10 min or until granules were fully hydrated. A second mixture of 24 ug purified G0RMI3 protein and 30 g of whole milk was mixed in an IKA Ultra Turrax TubeDrive (IKA Works, Wilmington, NC) for 3 min at 2,000 rpm. An 18.5 g aliquot of the first mixture was added to the second mixture (final concentration of 0.025% w/w/yogurt culture), and the combined mixture was mixed in the IKA Ultra Turrax TubeDrive for 1 min at 2000 rpm. The sample was then poured into a clean 80 mL glass Weck jar and seaed1 with 60 mm lid and gasket, and incubated at 44° C. for 4 hours (or until a pH of 4.6+/−0.1 was reached) to obtain a yogurt. After 24 hours, after 3-4 days, and after 6-8 days, the yogurt was evaluated by sensory experts for the presence of rancid smell and/or taste. From the 24-hour time point onwards, yogurts produced using the G0RMI3 protein powders were judged as rancid.

### Example 8: Removal of Esterase Activity Via Genetic Modification

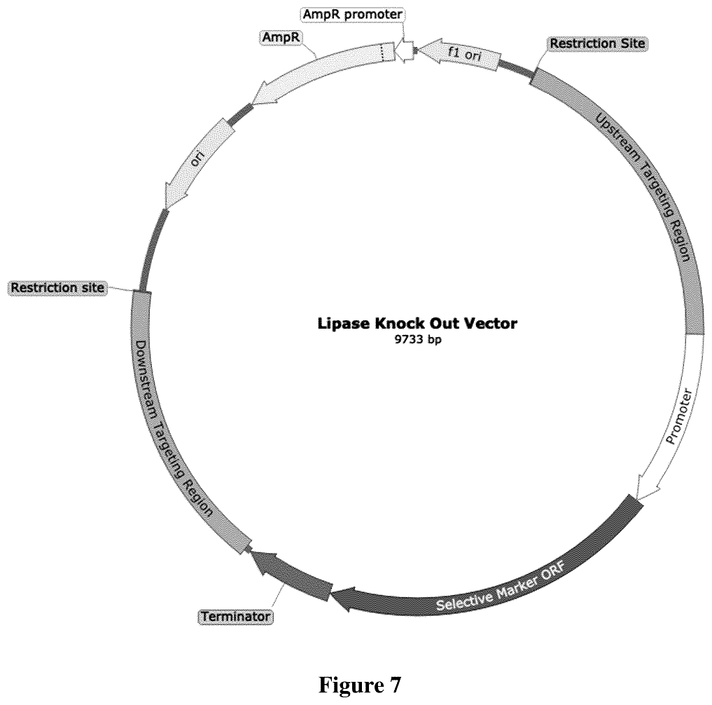

Recombinant Trichoderma reesei host cells capable of producing a recombinant β-lactoglobulin and comprising essentially eliminated FFA releasing activities of G0RH85 and G0RMI3 (double deficient); G0RH85, G0RMI3, and G0RGQ0 (triple deficient); G0RH85, G0RMI3, and G0RLH4 (triple deficient); G0RH85, G0RMI3 and G0RLH4 (triple deficient); and G0RH85, G0RMI3, G0RGQ0, and G0RLH4 (quadruple deficient) were generated by transforming protoplasts of a Trichoderma reesei strain capable of producing a recombinant β-lactoglobulin (“corresponding recombinant host cell”) with polynucleotides (targeting vectors) engineered to integrate by homologous recombination a selectable marker into genes encoding G0RH85, G0RMI3, G0RGQ0, and/or G0RLH4 proteins. The general structure of the targeting vector is shown in FIG. 7. The targeting vector comprised a selective marker (pyr4 gene, which enables growth without uracil supplementation) flanked by polynucleotide sequences that are homologous to the upstream and downstream polynucleotide sequences flanking the genes open reading frame of each gene in the Trichoderma reesei genome. Multiple gene replacements were completed by targeting the loci sequentially, and by recycling marker after each round.

Transformants were selected on minimal media, and then screened by PCR to identify a lipase knockout recombinant host cell. Lipase knock out recombinant host cells were “cured” of the selective marker by plating cells to 5-fluoroorotic acid containing media. “Cured” cells were taken for sequential rounds of transformation/curing until a strain in which all four gene open reading frames were knocked out was obtained.

The final knockout recombinant host cell and the corresponding recombinant host cell were fermented in a culture medium suitable for growth of the recombinant host cells and for production and secretion of the recombinant β-lactoglobulin.

What is claimed is:

1-44. (canceled)

45. A recombinant host cell capable of producing a recombinant component, wherein compared to a corresponding recombinant host cell the recombinant host cell comprises a decreased production and/or activity of a FFA releasing enzyme selected from the group consisting of FFA releasing enzymes comprising UniProt sequence #G0RH85, G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, G0RVD2, and G0R8A6, and homologs thereof, and combinations thereof.

46. The recombinant host cell of claim 45, wherein the FFA releasing enzyme consists of a FFA releasing enzyme selected from the group consisting of FFA releasing enzymes comprising UniProt sequence #G0RGQ0, G0RLH4, G0RMI3, and G0RGQ0, and combinations thereof.

47. The recombinant host cell of claim 45, wherein the FFA releasing enzyme consists of a first FFA releasing enzyme comprising UniProt sequence #G0RH85 and of a second FFA releasing enzyme selected from the group consisting of FFA releasing enzymes comprising UniProt sequence #G0RGQ0, G0RLH4, G0RMI3, and G0RGQ0, and combinations thereof.

48. The recombinant host cell of claim 45, wherein the decreased production and/or activity of the FFA releasing enzyme is a decreased production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

49. The recombinant host cell of any of claim 45, wherein the recombinant host cell is derived from a filamentous fungus selected from Aspergillus (e.g., Aspergillus niger), Trichoderma (e.g., Trichoderma reesei, Trichoderma citrinoviride), or Myceliophthora (e.g., Myceliophthora thermophila).

50. A method for producing a recombinant component, wherein the method comprises modulating production and/or activity of a FFA releasing enzyme selected from the group consisting of FFA releasing enzymes comprising UniProt sequence #G0RH85, G0R6T6, G0R6X2, G0R707, G0R7K1, G0R810, G0R9D1, G0R9F9, G0R9J9, G0R9X3, G0RBG0, G0RBJ0, G0RBM4, G0RBZ6, G0RD16, G0RDK5, G0RDU7, G0REM9, G0REZ4, G0RFR3, G0RFT3, G0RG04, G0RG60, G0RGD5, G0RGN7, G0RGQ0, G0RGQ7, G0RHJ4, G0RI29, G0RIJ9, G0RIU1, G0RIV5, G0RJ76, G0RJC6, G0RJY0, G0RK83, G0RKE6, G0RKH7, G0RKI9, G0RKL4, G0RL87, G0RLB0, G0RLB7, G0RLH4, G0RLL0, G0RLR3, G0RM14, G0RME5, G0RMI3, G0RNF8, G0RPQ8, G0RQD1, G0RQG3, G0RQJ5, G0RQN5, G0RR42, G0RRK3, G0RRQ4, G0RSK7, G0RTR6, G0RTT4, G0RUI0, G0RUZ9, G0RV93, G0RW73, G0RW77, G0RWS1, G0RWT9, G0RWY5, G0RX82, G0RX90, G0RHQ7, G0RVD2, and G0R8A6, and homologs thereof, and combinations thereof.

51. The method of claim 50, wherein the decreasing production and/or activity of the FFA releasing enzyme is a decreasing production and/or activity of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

52. The method of claim 50, wherein the decreasing production and/or activity of a FFA releasing enzyme is selected from: i) culturing a recombinant host cell capable of producing the recombinant component under fermentation conditions suitable for decreasing production and/or activity of the FFA releasing enzyme activity; ii) decreasing activity of the FFA releasing enzyme in a fermentation broth, a preparation, or a composition; iii) purifying the recombinant component away from the FFA releasing enzyme activity and/or purifying the FFA releasing enzyme activity away from the recombinant component; iv) fermenting the recombinant host cell of claim 1 in a culture medium under conditions suitable for production of the recombinant component; and combinations thereof.

53. The method of claim 52, wherein the method comprises adding to the fermentation broth, preparation, or composition an inhibitor of a FFA releasing enzyme that comprises a serine residue in its catalytic domain.

54. The method of claim 52, wherein the method comprises purifying the FFA releasing enzyme away from the recombinant component using an activity-based protein profiling (ABPP) small-molecule probe.

55. The method of claim 52, wherein the recombinant component is a recombinant milk protein.
